(19) United States
(12) Patent Application Publication
(10) Pub. No.: US 2025/0169491 A1
(43) Pub. Date: May 29, 2025

(54) POLYMERIZED HEMOGLOBIN SIZE FRACTIONATED VIA TANGENTIAL FLOW FILTRATION WITH LOW AUTO OXIDATION RATES

(71) Applicant: Ohio State Innovation Foundation, Columbus, OH (US)

(72) Inventors: Andre PALMER, Columbus, OH (US); Xiangming GU, Columbus, OH (US); Alisyn GREENFIELD, Columbus, OH (US); Clayton CUDDINGTON, Houston, TX (US)

(21) Appl. No.: 18/842,276

(22) PCT Filed: Aug. 26, 2022
(86) PCT No.: PCT/US2022/041743

§ 371 Date: Aug. 28, 2024

Related U.S. Application Data
(60) Provisional application No. 63/327,979, filed on Apr. 6, 2022.
(60) Provisional application No. 63/314,843, filed on Feb. 28, 2022.

(51) Int. Cl. A01N 1/126; A01N 1/125; B01D 61/14; B01D 69/02

(52) U.S. Cl. CPC A01N 1/126; A01N 1/125; B01D 61/145; B01D 69/02; B01D 2315/10; B01D 2317/022; B01D 2317/025; B01D 2325/34

(57) ABSTRACT

Due to a rising demand in the need for organ transplantation and a critical donor organ shortage, the need to fill this gap has increased the use of ECD and donation after cardiac death (DCD) organs viable for transplantation to lower the mortality of patients on the organ waiting list. Described herein is a perfusion solution comprising polymerized hemoglobin, wherein the perfusion solution comprises less than 5% by weight low molecular weight hemoglobin species, based on the total weight of the perfusion solution.

### CROSS-REFERENCE TO RELATED APPLICATIONS

This application claims benefit of U.S. Provisional Application No. 63/314,843, filed Feb. 28, 2022, and U.S. Provisional Application No. 63/327,979, filed Apr. 6, 2022, each of which is hereby incorporated herein by reference in its entirety.

### ACKNOWLEDGEMENT OF GOVERNMENT SUPPORT

This invention was made with government support under Grant No. W81XWH-18-1-0059 awarded by the Department of Defense, and Grant Nos. R01HL126945, R01HL138116, R01HL156526, and R01EB021926 awarded by the National Institutes of Health. The government has certain rights in the invention.

### BACKGROUND

In the United States in 2020, there were almost 108,000 patients on the organ transplant waiting list, yet only 39,000 transplants were performed leading to 11,000 patients being removed from the waiting list due to death or sickness. A primary reason for the disparity between the number of patients on the waiting list and the number of transplants performed is an overall limited number of available organs and additionally some of the available supply of organs are deemed of poor quality and are not viable for transplantation. For organs such as the lungs, more than 80% of donor allografts offered by organ donors are declined due to poor organ quality. It is obvious that the best way to decrease the number of patients on the organ waiting list is to make more organs available for transplantation. In an effort to expand the donor pool, organs from marginal or extended criteria donors (ECD) may be evaluated. However, ECD grafts are associated with a significantly higher risk of ischemia-reperfusion injury (IRI) which leads to primary graft dysfunction and subsequently reduces organ viability. Due to a rising demand in the need for organ transplantation and a critical donor organ shortage, the need to fill this gap has increased the use of ECD and donation after cardiac death (DCD) organs viable for transplantation to lower the mortality of patients on the organ waiting list.

### SUMMARY

In accordance with the purposes of the disclosed materials and methods, as embodied and broadly described herein, the disclosed subject matter, in one aspect, relates to a perfusion solution for tissue recovery. perfusion solution can comprise polymerized hemoglobin, wherein the perfusion solution comprises less than 5% by weight low molecular weight hemoglobin species, based on the total weight of the perfusion solution.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate certain examples of the present disclosure and together with the description, serve to explain, without limitation, the principles of the disclosure. The application includes reference to the accompanying figures, in which:

FIG. 1 shows an exemplary NEVLP circuit is shown, wherein a sweep gas across the oxygenator is composed of 8%/6%/86% CO₂/O₂/N₂. A heat exchanger maintains the perfusate reservoir and flow path at 37° C.

FIG. 2 is an exemplary graph of the concentration of PolyhHb in the HBOC perfusate and the corresponding hematocrit of RBCs in the perfusate during NEVLP. The hematocrit decreases as a function of time for RBCs, which signifies hemolysis occurring during NEVLP. The hematocrit equivalent for PolyhHb is calculated by multiplying the concentration in g/dL by 3.

FIG. 3 is an exemplary graph of the percent change in metHb level of the synthesized PolyhHb. In this embodiment, the percent change in metHb level is lower and more controlled than the empty circuit HBOC NMP embodiments. PolyhHb even experienced a lower rate of auto-oxidation compared to the RBC perfusate due to the absence of cell-free Hb in solution.

FIG. 4 is an exemplary average SEC-HPLC chromatogram for the PolyhHb perfusate both before and after perfusion. The size distributions overlapping each other demonstrate that the PolyhHb remains structurally intact during NEVLP.

FIG. 5 is an exemplary graph of pO₂ of the perfusate exiting the lung. PolyhHb exhibited higher post-lung pO₂s compared to RBCs after 30 minutes and compared to both perfusates by 90 minutes. The PolyhHb perfusate was also the only one to not significantly decrease in oxygenation over time.

FIG. 6 is an exemplary graph of the amount of O₂ delivered to the perfusate by the lung. RBCs exhibited lower oxygenation capacity compared to both other perfusates after 30 minutes. The RBC perfusate was also the only one to decrease in the amount of oxygen delivered over time.

FIG. 7 is an exemplary graph of the change in partial pressure of CO₂ (pCO₂) of the perfusate across the lung. Only the RBC perfusate did not have a decrease in CO₂ clearance.

FIG. 8 is an exemplary graph of PA pressure over the course of NEVLP. PA pressure for the RBC perfusates was significantly higher than all other groups across all time points. In these embodiments, three of the six RBC perfusions had to be cut short after 60 minutes due to the development of PA pressures over 100 cm H₂O.

FIG. 9 is an exemplary graph of PVR over time, which in this embodiment, shows a similar trend to PA pressure where the RBC perfusate yielded higher values compared to the PolyhHb or control perfusate.

FIG. 10 is an exemplary graph of the change in lung weight over time. In this embodiment at 60 minutes, the value for the RBC perfusates spiked due to three lungs that were then deemed non-viable and removed from the circuit. While the PolyhHb and control perfusates seem to stabilize, the RBC perfusates demonstrate a continued increase in lung weight, signifying continuing tissue damage and edema.

FIG. 11 is an exemplary graph of the change in LDH level of the various perfusates during perfusion. The PolyhHb perfusate was the only group to not achieve a significantly higher LDH release compared to the 30 minute timepoint. It also demonstrated significantly less LDH release compared to the RBC perfusate by 60 minutes and compared to both the RBC and control perfusates by 90 minutes. All of these findings point to less cellular damage from the PolyhHb perfusate compared to the other groups.

FIG. 12 is an exemplary graph of the wet/dry ratio for three perfusates: colloid control, RBC, and polymerized human hemoglobin. The polymerized human hemoglobin perfusate exhibited less tissue edema in comparison to the colloid control and RBC perfusates.

FIGS. 13A and 13B show (13A) the biophysical parameters of the PolyhHb synthesized at the pilot scale using a 30:1 molar ratio of glutaraldehyde to hHb compared to previous generations of exemplary commercial HBOCs and (13B) the biophysical properties for the various exemplary perfusates, wherein the hematocrit equivalent of PolyhHb was found by multiplying the PolyhHb concentration in g/dL by 3.

FIGS. 14A and 14B show lung tissue analysis of iron and Hb. FIG. 14A shows lung tissue iron as measured by the ferrozine assay, which shows the mean±S.D. for total iron quantified from tissue perfused with a control, RBCs and PolyhHb (n=6/group). FIG. 14B shows Hb immunohistochemistry of lung tissue sections after perfusion with the control perfusate at (a) 50 times magnification and (b) 630 times magnification. Lungs perfused with RBCs are shown in (c) at 50 times magnification with multiple vessels staining positive for Hb (black asterisks) and (d) at 630 times PolyHb magnification with vessel lumen Hb localized to endothelium (white asterisks). Lung tissue perfused with PolyhHb is shown in (e) at 50 times magnification and (f) at 630 times magnification showing adventitia Hb (black asterisk). Scale bars represent 500 microns (50 times magnification, images a, c, e) and 30 microns (630 times magnification, images b, d, f). Statistical analysis was performed using a One-way ANOVA with a Holm-Sidak's multiple comparisons test based on a normal distribution of data (Kolmogorov-Smirnov test). Significance was set at a p<0.05 and p values are reported as actual values to two significant figures. All analyses were performed using GraphPad Prism 9.1.1 (San Diego, California). For all images, regions of interest correspond to the same vessels in tissue sections separated by 5-micron tissue cuts. In these sections, most of the Hb from RBCs or PolyhHb would be expected to accumulate within the intravascular lumen or regions (adventitial—vasculature and macrophages) surrounding pre- and post-capillary pulmonary vessels. As expected, Hb-specific immunohistochemistry does not reveal visual reactivity within or around vessels of control perfused lung at 50 times or 630 times magnification, as shown in FIG. 14B(a) and (b). In contrast to the control perfused lungs, 14B(c) and (d) show that RBC perfused tissue demonstrates diffuse reactivity for Hb within the vascular lumen of multiple vessels (black asterisk) at 50 times magnification and at 630 times magnification near the vascular endothelium indicating a substantial build-up in cell-free Hb. Conversely, PolyhHb perfused lung tissue show visually less intra-vascular hemoglobin accumulation at 50 times magnification, but mild adventitial Hb (black asterisk) at 630 times magnification in 14B(e) and (f) respectively. This further shows the superiority of PolyhHb as the significant reduction in iron and Hb deposition compared to RBC perfused lungs demonstrates the reduced heme cytotoxicity of the next-generation PolyhHb perfusate.

FIG. 15 shows that for the PolyhHb and asanguinous perfusates, the K⁺ concentration of the inlet was lower than the outlet. Initially this can be explained by the Perfadex flush step that happened before NEVLP began because Perfadex has a [K⁺] of 5 mM, so residual amounts of Perfadex being removed from the organ carried a higher amount of K⁺. The opposite was true for the RBC perfusate. The RBC storage solution had a higher [K⁺] than Perfadex so the initial outlet [K⁺] was marginally lower than the inlet. The increase in [K⁺] of the RBC storage solution also explained the significantly higher K concentration compared to the other two perfusates.

FIG. 16 shows that the [Na⁺] of all exemplary species increased insignificantly over the NEVLP. Additionally, [Na+] of the PolyhHb was marginally higher than the other perfusates but this was easily explained by the fact that the PolyhHb storage buffer—modified Ringer's lactate—had an [Na⁺] of 155 mM compared to 140-145 mM for William's media FIG. 17 shows that [Ca²⁺] remained relatively constant throughout the NEVLP. The outlet Ca²⁺ concentrations of the PolyhHb and asanguinous perfusates were lower than the inlet concentrations but this was not unexpected as increased cellular uptake of Ca²⁺ post-ischemia had been shown previously. The RBC perfusate demonstrated significantly higher [Ca²⁺] compared to the other perfusates likely due to a higher Ca²⁺ concentration in residual RBC storage buffer.

FIG. 18 shows that there was no difference between inlet and outlet Cl⁻ concentrations for any perfusate. The PolyhHb and asanguinous perfusates both remained constant throughout the NEVLP; however, the RBC perfusate demonstrated an uptick in [Cl⁻] after 60 minutes possibly correlated to the decline in lung health seen in other organ metrics at this time.

FIG. 19 shows that all three perfusates maintained a steady glucose concentration during the NEVLP. The asanguinous perfusate was the only one that demonstrated an increase in glucose concentration across the organ possibly because glucose consumption was an aerobic process and in a state of hypoxia without an O₂ carrier present, glucose uptake is significantly reduced. The PolyhHb perfusate had the lowest glucose concentration because it had the least amount of William's media in it and William's media was the sole source of glucose for these experiments.

FIG. 20 shows that both the RBC and PolyhHb perfusates showed trends for increasing lactate concentrations during the NEVLP, but only the RBC perfusate increased significantly compared to the t=0 point. This validates the results seen in the LDH results where all perfusates saw an increase in LDH, but the PolyhHb perfusate did not demonstrate a significant increase in LDH. The asanguinous control is not included in this figure because there were not enough lactate concentration data points above the lower limit of the blood gas analyzer (0.4 mM).

FIG. 21 shows that the pH of the perfusate at the outlet slightly decreased for all three perfusate between 0 and 60 minutes. After 60 minutes, the pH was stable for both the PolyhHb and the asanguinous perfusate, but the RBC perfusate began increasing in pH after this time.

By the end of the NEVLP, the pH of the RBC perfusate was significantly higher than the other two perfusates. Given that lungs are supposed to be mildly acidotic, the increase in pH towards 7.4 corroborates the decline in lung function seen by other metrics at this time.

FIGS. 22A-22D show the (FIG. 22A) O₂ equilibrium curve and the (FIG. 22B) O₂ offloading kinetics for PolyhHb-B1, PolyhHb-B2, PolyhHb-B3, PolyhHb-B4 and hHb. Lines represent the mean from all batches. Shaded areas indicate the standard error for each PolyhHb bracket. FIG. 22C shows the pseudo-first-order Hp binding kinetics of PolyhHb/hHb. The normalized fluorescence changes (λ_{excitation}=285 nm, λₑₘᵢₛₛᵢₒₙ=310 nm) were fit to a monoexponential equation. FIG. 22D shows the second order Hp binding kinetics of PolyhHb-B1, PolyhHb-B2, PolyhHb-B3, PolyhHb-B4 and hHb. The second order Hp binding rate constant was obtained via performing a linear fit of the pseudo-first-order Hp binding rate constant to the Hb concentration.

FIGS. 23A-23C show the size and MW distribution of PolyhHb and hHb. FIG. 23A shows the normalized intensity distribution of the elution time for hHb, PolyhHb-B1, PolyhHb-B2, PolyhHb-B3, and PolyhHb-B4 measured using SEC-HPLC. Distributions were taken from the 413 nm absorbance normalized against the maximum intensity. FIG. 23B shows the effective diameters of hHb, PolyhHb-B1, PolyhHb-B2, PolyhHb-B3, and PolyhHb-B4, measured using DLS at λ=632 nm and a 90-degree angle. FIG. 23C shows the denaturing SDS-PAGE (Novex™ (10-20%) Tris-glycine gel) of hHb, PolyhHb-B1, PolyhHb-B2, PolyhHb-B3, and PolyhHb-B4, wherein DLS stands for dynamic light scattering, SDS-PAGE stands for sodium dodecyl sulfate polyacrylamide gel electrophoresis; SEC-HPLC stands for size exclusion high performance liquid chromatography, PolyhHb stands for polymerized human hemoglobin, and hHb stands for human hemoglobin.

FIG. 24 shows the biophysical properties of hHb and PolyhHb fractions, including properties related to O₂ binding, size, and Hp binding kinetics. Measured characteristics reported as average±standard deviation.

FIG. 25 shows an exemplary diagram of the cell wash and hHb purification process. RBCs were first washed over a 0.65 μm filter with cell-free hHb and cell debris permeating into the waste. After 6 diacycles of cell wash, the vessel was filled with PB to lyse the RBCs. The lysate was then passed through the 500 kDa filter to obtain purified hHb, which was then used to polymerize hHb.

FIG. 26 shows a diagram of an exemplary reactor system used for the hHb polymerization process. The reactor was filled with purified hHb on day 1, polymerized with glutaraldehyde and subsequently quenched with NaCNBH₃ on day 2 and transferred into the PolyhHb TFF purification process on day 3.

FIG. 27 shows a diagram of an exemplary 2-stage TFF PolyhHb purification process. Stage 1 retains any polymers that are too large (>0.2 μm), and stage 2 facilitates removal of unreacted chemicals and LMW Hb species from the system (<500 kDa). The PolyhHb is washed with >12 diacycles of a modified Ringer's lactate solution to remove the majority of LMW species and to buffer exchange the PolyhHb into the modified Ringer's lactate solution.

FIGS. 28A and 28B show exemplary geometry and meshing of the continuous stirred tank reactor generated by Comsol 5.3a for the PolyhHb reactor.

FIGS. 29A and 29B show the Hb concentration during the TFF-facilitated RBC washing process. Concentration of cell-free Hb in the permeate stream (29A) and concentration of cell-free Hb in the retentate vessel (29B) monitored over 6 diacycles of the RBC washing process. The cell-free Hb concentration decreased significantly over 4 diacycles in both the retentate and the permeate and remained constant from diacycle 4 to 6. Outliers are shown as black squares. A total of 5 replicates were used.

FIGS. 30A and 30B show the HCT and RBC concentration during the TFF-facilitated RBC washing process. HCT of the pooled RBC solution (30A) and cell concentration (30B) over 6 diacycles of the RBC washing process. For both the HCT and cell concentration, there was no significant difference between diacycles, indicating that there was no significant cell lysis throughout the RBC washing process. Outliers are shown as black squares. A total of 5 replicates were used.

FIG. 31 shows an exemplary OEC displaying the O₂ saturation of hHb and PolyhHb as a function of O₂ tension. The moderate P₅₀ pilot scale PolyhHb batches exhibited a significantly higher O₂ affinity compared to fully T-state pilot scale batches and bench-top scale 30:1 T-state PolyhHb batches. The OECs are displayed as averages+/−one standard deviation shown in grey. All measurements were taken at 37° C. and pH 7.4.

FIG. 32 shows exemplary SEC-HPLC elution curves for the three types of PolyhHb discussed in this study. Bench-top scale PolyhHb batches exhibited a lower elution time, but there was a noticeable peak at 10 minutes corresponding to residual cell-free Hb, which is more completely eliminated in pilot scale batches.

FIG. 33 shows the auto-oxidation kinetics of hHb and PolyhHb. The decrease in [Fe²⁺] was linearized according to first-order rate kinetics. T-state PolyhHb—regardless of scale—exhibited a kₒₓ ˜4× higher than that of the moderate P₅₀ pilot scale PolyhHb. There was no significant difference in kₒₓ between pilot and bench-top scale T-state PolyhHb.

FIG. 34 shows exemplary O₂ offloading kinetics of hHb and PolyhHb. While T-state PolyhHb trended towards a higher k_{O2,off}, and moderate P₅₀ PolyhHb trended towards a lower k_{O2,off}, there was no correlation found in this study. Bench-top scale T-state PolyhHb had a significantly higher k_{O2,off} than both pilot scale groups.

FIGS. 35A and 35B show Hp binding kinetics to hHb and PolyhHb. Representative first order kinetics for the reaction of 0.25 μM Hp with 1.25 μM PolyhHb (35A) and linearization of the apparent first order rate constant as a function of [Hb](35B). There was no significant difference between any groups or batches of PolyhHb in either the first or second order rate kinetics. All measurements were taken at a 285 nm emission.

FIGS. 36A-36F show various turbulence parameters with streamlines in the continuous stirred tank reactor vessel. Dynamic viscosity (36A), eddy diffusivity (36B), pressure (36C), Reynold's number (36D), shear rate (36E), and vorticity (36F) in the reactor vessel were all modeled using computational fluid dynamics (CFD).

FIG. 37 shows the biophysical properties of 8 batches of pilot scale PolyhHb produced: Pilot T-state, Pilot Moderate P₅₀, Bench-top PolyhHb, and Unmodified hHb.

Reference will now be made in detail to specific aspects of the disclosed materials, compounds, compositions, formulations, articles, and methods, examples of which are illustrated in the accompanying Examples and Figures.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### General Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used in the specification and the appended claims, the singular forms “a,” “an” and “the” include plural referents unless the context clearly dictates otherwise. Thus, for example, reference to “a compound”, “a composition”, or “a disorder”, includes, but is not limited to, two or more such compounds, compositions, or disorders, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise. As used herein, the term “substantially free,” when used in the context of a composition or component of a composition that is substantially absent, is intended to refer to an amount that is then about 1% by weight or less, e.g., less than about 0.5% by weight, less than about 0.1% by weight, less than about 0.05% by weight, or less than about 0.01% by weight of the stated material, based on the total weight of the composition.

As used herein, “molecular weight” refers to the sum of the atomic masses of all atoms in a molecule, based on a scale in which the atomic masses of hydrogen, carbon, nitrogen, and oxygen are 1, 12, 14, and 16, respectively.

As used herein, “albumin” means a small globular protein with a molecular weight of 66.5 kilodaltons (kDa). It consists of 585 amino acids which are organized into three repeated homologous domains and are made up of two separate sub-domains, A and B.

As used herein, “osmolarity” refers to the concentration of a solution expressed as the total number of solute particles per liter.

As used herein, “viscosity” refers to a quantity expressing the magnitude of internal friction, as measured by the force per unit area resisting a flow in which parallel layers unit distance apart have unit speed relative to one another.

As used herein, “colloid osmotic pressure” refers to the physiochemical phenomenon that occurs when two solutions with different colloid concentrations are separated by a semipermeable membrane. It is a type of osmotic pressure induced by colloids, which can include protein and more specifically albumin, in a blood vessel's plasma that cause a pull on fluid back into the capillary.

As used herein, “glutaraldehyde” refers to C₅H₈O₂ or OCH(CH₂)₃CHO, is a transparent oily, liquid with a pungent odor. It is a dialdehyde comprised of pentane with aldehyde functions at C-1 and C-5. Alternatives to glutaraldehyde can include carboiimide, diisocyanates and polyepoxy compounds, as well as Genipin (Challenge Bioproducts Co., Ltd., Taiwan), epigallocatechin gallate (Sigma, St. Louis, MO), and grape seed proanthocyanidin (PureBulk, Inc., Roseburg, OR).

As used herein, “normothermic conditions” refers to a condition of normal body temperature. In some embodiments, normothermic conditions can include a temperature from 36° C. to 38° C.

### Composition

The present disclosure provides for a perfusion solution for tissue recovery. The perfusion solution can comprise polymerized hemoglobin and less than 5% by weight low molecular weight hemoglobin species, based on the total weight of the perfusion solution.

As used herein, “polymerized hemoglobin”, also referred to as “polymerized Hb” or “PolyhHb”, refers to a class of hemoglobin (Hb) based O₂ carrier (HBOC) that can be synthesized and purified at large scale such that it can transport and offload O₂ to support cellular metabolism, while not demonstrating cytotoxic side-effects. In some embodiments, hemoglobin is polymerized with glutaraldehyde. As used herein, “hemoglobin species, or “hemoglobin (Hb)”, refers to the protein inside red blood cells that carries oxygen from the lungs to tissues and organs in the body and carries carbon dioxide back to the lungs. It can include four protein chains, two alpha chains and two beta chains, each with a ring-like heme group containing an iron atom. Oxygen can bind reversibly to these iron atoms and can be transported through blood.

As used herein, “perfusion solution”, also referred to as “perfusate”, refers to the solution used to perfuse a tissue sample during perfusion. In some embodiments, a perfusion solution can include PolyhHb diluted with William's cell culture media. In further embodiments, the perfusion solution can include albumin (e.g., human serum albumin). In some embodiments, the perfusion solution can include from 1 to 15 g/dL polymerized hemoglobin, 25 to 85 mM NaCl, 1 to 3 mM KCl, 6 to 20 mM KH₂PO₄, 20 to 70 mM sodium gluconate, 5 to 21 mM sodium lactate, 1 to 4 mM magnesium gluconate, 0.6 to 1.2 mM CaCl₂ dihydrate, 11 to 16 mM NaOH, 1 to 4 mM adenine, 2 to 8 mM dextrose, 0.5 to 3 mM glutathione, 2 to 8 mM HEPES, 1 to 4 mM ribose, 7 to 30 mM mannitol, 10 to 40 g/L hydroxyethyl starch, and 40 to 160 mg/dL N-acetyl-L-cysteine. In further embodiments, the perfusion solution can include from 3 to 4 g/dL polymerized hemoglobin, 25 to 85 mM NaCl, 1 to 3 mM KCl, 6 to 20 mM KH₂PO₄, 20 to 70 mM sodium gluconate, 5 to 21 mM sodium lactate, 1 to 4 mM magnesium gluconate, 0.6 to 1.2 mM CaCl₂) dihydrate, 11 to 16 mM NaOH, 1 to 4 mM adenine, 2 to 8 mM dextrose, 0.5 to 3 mM glutathione, 2 to 8 mM HEPES, 1 to 4 mM ribose, 7 to 30 mM mannitol, 10 to 40 g/L hydroxyethyl starch, and 40 to 160 mg/dL N-acetyl-L-cysteine.

As used herein, “hemoglobin species, or “hemoglobin (Hb)”, refers to the protein inside red blood cells that carries oxygen from the lungs to tissues and organs in the body and carries carbon dioxide back to the lungs. It can include four protein chains, two alpha chains and two beta chains, each with a ring-like heme group containing an iron atom. Oxygen can bind reversibly to these iron atoms and can be transported through blood.

In some embodiments, the low molecular weight hemoglobin species can have a molecular weight below 500 kDa (e.g., a molecular weight below 300 kDa). In some embodiments, the low molecular weight hemoglobin species can have a molecular weight from below 500 kDa to 400 kDa, from 400 kDa to 300 kDa, from 300 kDa to 200 kDa, from 200 kDa to 100 kDa, from 100 kDa to 50 kDa, or from 50 kDa to above 0 kDa.

In some embodiments, the low molecular weight hemoglobin species includes unreacted hemoglobin. As used herein, “unreacted hemoglobin” refers to hemoglobin that has not reacted or bound to another molecule and therefore has a low molecular weight. Unreacted hemoglobin, like cell-free hemoglobin, can extravasate out of circulation into tissue space, thereby causing nitric oxide scavenging and subsequently vasoconstriction, systemic hypertension, and/or oxidative tissue injury.

In some embodiments, the low molecular weight hemoglobin species can include cell-free hemoglobin. As used herein, “cell-free hemoglobin” refers to hemoglobin molecules that have been separated from the red blood cells within which they originally occurred, often through the process of hemolysis.

In some embodiments, the perfusion solution comprises less than 5% by weight (e.g., less than 4% by weight, less than 3% by weight, less than 2% by weight, less than 1% by weight, or less than 0.5% by weight) hemoglobin species having a molecular weight below 500 kDa, based on the total weight of the perfusion solution.

In some embodiments, the perfusion solution comprises less than 5% by weight (e.g., less than 4% by weight, less than 3% by weight, less than 2% by weight, less than 1% by weight, or less than 0.5% by weight) hemoglobin species having a molecular weight below 300 kDa, based on the total weight of the perfusion solution.

As used herein, “filter” can include tangential-flow filtration. The term “tangential-flow filtration” refers to a process in which the fluid mixture containing the components to be separated by filtration is recirculated at high velocities tangential to the plane of the filtration membrane to reduce fouling of the filter. In such filtrations a pressure differential is applied along the length of the filtration membrane to cause the fluid and filterable solutes to flow through the membrane (e.g., filter). This filtration is suitably conducted as a batch process as well as a continuous-flow process. For example, the solution may be passed repeatedly over the membrane while that fluid which passes through the filter is continually drawn off into a separate unit or the solution is passed once over the membrane and the fluid passing through the filter is continually processed downstream. As used herein, “filtration membrane” refers to microporous barriers of, for example, polymeric, ceramic, or metallic materials which are used to separate dissolved materials (solutes), colloids, or find particular from solutions. A filtration membrane can be rated for retaining solutes have a specific molecular weight range from the molecular weight of one component in the solution to another component in the solution. By way of example, a filtration membrane can be rated for retaining polymerized hemoglobin with a molecular weight above that of a low molecular weight hemoglobin species (e.g., such as a membrane rated for retaining solutes have a molecular weight above 300 kDa).

In some embodiments, the polymerized hemoglobin can have an average molecular weight from 300 kDa to 500 kDa and can be filtered using a filtration membrane with a cutoff value from 300 kDa to 500 kDa. In further embodiments, the polymerized hemoglobin can have an average molecular weight from 300 kDa to 350 kDa, 350 kDa to 400 kDa, 400 kDa to 450 kDa, or 450 kDa to 500 kDa and the cutoff value can be from 300 kDa to 350 kDa, 350 kDa to 400 kDa, 400 kDa to 450 kDa, or 450 kDa to 500 kDa.

In some embodiments, the polymerized hemoglobin can have an average molecular weight from 500 kDa to 750 kDa and can be filtered using a filtration membrane with a cutoff value from 500 kDa to 750 kDa. In further embodiments, the polymerized hemoglobin can have an average molecular weight from 500 kDa to 550 kDa, 550 kDa to 600 kDa, 600 kDa to 650 kDa, 650 kDa to 700 kDa, or 700 kDa to 750 kDa and the cutoff value can be from 500 kDa to 550 kDa, 550 kDa to 600 kDa, 600 kDa to 650 kDa, 650 kDa to 700 kDa, or 700 kDa to 750 kDa.

In some embodiments, the polymerized hemoglobin can have an average molecular weight from 750 kDa to 50 nm and can be filtered using a filtration membrane with a cutoff value from 750 kDa to 50 nm. In further embodiments, the polymerized hemoglobin can have an average molecular weight from 750 kDa to 800 kDa, 800 kDa to 850 kDa, 850 kDa to 900 kDa, 900 kDa to 950 kDa, 950 kDa to 1000 kDa, 1000 kDa to 25 nm, or 25 nm to 50 nm and the cutoff value can be from 750 kDa to 800 kDa, 800 kDa to 850 kDa, 850 kDa to 900 kDa, 900 kDa to 950 kDa, 950 kDa to 1000 kDa, 1000 kDa to 25 nm, or 25 nm to 50 nm.

In some embodiments, the polymerized hemoglobin can have an average molecular weight from 50 nm to 0.2 μm and can be filtered using a filtration membrane with a cutoff value from 50 nm to 0.2 μm. In further embodiments, the polymerized hemoglobin can have an average molecular weight from 50 nm to 100 nm, 100 nm to 150 nm, or 150 nm to 0.2 μm and the cutoff value can be from 50 nm to 100 nm, 100 nm to 150 nm, or 150 nm to 0.2 μm.

In some embodiments, the polymerized hemoglobin can be prepared by a process that includes polymerizing the hemoglobin and filtering the perfusion solution by ultrafiltration against a filtration membrane having a pore size that separates the low molecular weight hemoglobin species from the polymerized hemoglobin. As used herein, “filtration membrane” refers to microporous barriers of, for example, polymeric, ceramic, or metallic materials which are used to separate dissolved materials (solutes), colloids, or find particular from solutions. A filtration membrane can be rated for retaining solutes have a specific molecular weight range from the molecular weight of one component in the solution to another component in the solution. By way of example, a filtration membrane can be rated for retaining polymerized hemoglobin with a molecular weight above that of a low molecular weight hemoglobin species (e.g., such as a membrane rated for retaining solutes have a molecular weight above 300 kDa).

In further embodiments, polymerizing hemoglobin includes adding to a Hb solution a glutaraldehyde solution over a specified period of time. In certain embodiments, polymerizing hemoglobin includes adding to a Hb solution a 0.75 wt. % glutaraldehyde solution in phosphate buffered saline (0.1 M, pH 7.4) in a 30:1 molar ratio, glutaraldehyde to Hb, over a period of 3 hours and mixing the solution for an additional hour after the glutaraldehyde addition is completed.

In some embodiments, the filtration membrane can be rated for retaining solutes having a molecular weight greater than the molecular weight of the low molecular weight hemoglobin species, thereby forming a retentate fraction including the polymerized hemoglobin and a permeate fraction including the low molecular weight hemoglobin species. As used herein, a “retentate fraction” refers to the fraction of solution that is unable to pass through the filtration membrane. In some embodiments, the retentate fraction can include polymerized hemoglobin. As used herein, a “permeate fraction” refers to the fraction of solution that permeates the filtration membrane. In some embodiments, the permeate fraction can include low molecular weight hemoglobin species. In other embodiments, the permeate fraction can include polymerized hemoglobin and low molecular weight hemoglobin species.

In some embodiments, ultrafiltration can include tangential-flow filtration. As used herein, the term “tangential-flow filtration” refers to a process in which the fluid mixture containing the components to be separated by filtration is recirculated at high velocities tangential to the plane of the filtration membrane to reduce fouling of the filter. In such filtrations a pressure differential is applied along the length of the filtration membrane to cause the fluid and filterable solutes to flow through the membrane (e.g., filter).

This filtration is suitably conducted as a batch process as well as a continuous-flow process. For example, the solution may be passed repeatedly over the membrane while that fluid which passes through the filter is continually drawn off into a separate unit or the solution is passed once over the membrane and the fluid passing through the filter is continually processed downstream.

In some embodiments, the retentate fraction including polymerized hemoglobin can have a molecular weight of greater than 300 kDa and the permeate fraction including the low molecular weight hemoglobin species can have a molecular weight of less than 300 kDa. In certain embodiments, the polymerized hemoglobin can have a molecular weight from greater than 300 kDa to 500 kDa, 300 kDa to 750 kDa, 300 kDa to 50 nm, 300 kDa to 100 nm, 300 kDa to 0.2 μm, 500 kDa to 750 kDa, 500 kDa to 50 nm, 500 kDa to 100 nm, 500 kDa to 0.2 μm, 750 kDa to 50 nm, 750 kDa to 100 nm, 750 kDa to 0.2 μm, or 50 nm to 0.2 μm. In some embodiments, the low molecular weight hemoglobin species can have a molecular weight from below 300 kDa to 200 kDa, from 200 kDa to 100 kDa, from 100 kDa to 50 kDa, or from 50 kDa to above 0 kDa.

In some embodiments, the polymerized hemoglobin can be prepared by a process that can further include filtering the retentate fraction comprising the polymerized hemoglobin by ultrafiltration against a second filtration membrane, thereby forming a second retentate fraction comprising the polymerized hemoglobin with a molecular weight above a cutoff value and a second permeate fraction comprising species having a molecular weight below the cutoff value and above 300 kDa.

In some embodiments, the cutoff value can be from 300 kDa to 0.2 μm.

In some embodiments, the cutoff value can be from 300 kDa to 500 kDa. In further embodiments, the cutoff value can be from 300 kDa to 350 kDa, 350 kDa to 400 kDa, 400 kDa to 450 kDa, or 450 kDa to 500 kDa.

In some embodiments, the cutoff value can be from 500 kDa to 750 kDa. In further embodiments, the cutoff value can be from 500 kDa to 550 kDa, 550 kDa to 600 kDa, 600 kDa to 650 kDa, 650 kDa to 700 kDa, or 700 kDa to 750 kDa.

In some embodiments, the cutoff value can be from 750 kDa to 50 nm. In further embodiments, the cutoff value can be from 750 kDa to 800 kDa, 800 kDa to 850 kDa, 850 kDa to 900 kDa, 900 kDa to 950 kDa, 950 kDa to 1000 kDa, 1000 kDa to 25 nm, or 25 nm to 50 nm.

In some embodiments, the cutoff value can from 50 nm to 0.2 μm. In some embodiments, the cutoff value can be from 50 nm to 100 nm, 100 nm to 150 nm, or 150 nm to 0.2 μm.

In some embodiments, the polymerized hemoglobin can be prepared by a process that can further include filtering the second retentate fraction including the polymerized hemoglobin by ultrafiltration against a third filtration membrane, thereby forming a third retentate fraction comprising the polymerized hemoglobin with a molecular weight above a second cutoff value and a third permeate fraction including species having a molecular weight below the second cutoff value and above the cutoff value.

In some embodiments, the second cutoff value can be from the cutoff value to 0.2 μm.

In some embodiments, the second cutoff value can be from the cutoff value to 500 kDa. In some embodiments, the second cutoff value can be from the cutoff value to 350 kDa, 350 kDa to 400 kDa, 400 kDa to 450 kDa, or 450 kDa to 500 kDa.

In some embodiments, the second cutoff value can be from 500 kDa to 750 kDa. In some embodiments, the second cutoff value can be from 500 kDa to 550 kDa, 550 kDa to 600 kDa, 600 kDa to 650 kDa, 650 kDa to 700 kDa, or 700 kDa to 750 kDa.

In some embodiments, the second cutoff value can be from 750 kDa to 50 nm. In some embodiments, the second cutoff value can be from 750 kDa to 800 kDa, 800 kDa to 850 kDa, 850 kDa to 900 kDa, 900 kDa to 950 kDa, 950 kDa to 1000 kDa, 1000 kDa to 25 nm, or 25 nm to 50 nm.

In some embodiments, the second cutoff value can be from 50 nm to 0.2 μm. In some embodiments, the second cutoff value can be from 50 nm to 100 nm, 100 nm to 150 nm, or 150 nm to 0.2 μm.

In some embodiments, the polymerized hemoglobin can be prepared by a process that can further include filtering the third retentate fraction including the polymerized hemoglobin by ultrafiltration against a fourth filtration membrane, thereby forming a fourth retentate fraction comprising the polymerized hemoglobin with a molecular weight above a third cutoff value and a fourth permeate fraction including low molecular weight hemoglobin species having a molecular weight below the third cutoff value and above the second cutoff value.

In some embodiments, the third cutoff value can be from the second cutoff value to 0.2 μm.

In some embodiments, the third cutoff value can be from the second cutoff value to 500 kDa. In some embodiments, the third cutoff value can be from the second cutoff value to 350 kDa, 350 kDa to 400 kDa, 400 kDa to 450 kDa, or 450 kDa to 500 kDa.

In some embodiments, the third cutoff value can be from 500 kDa to 750 kDa. In some embodiments, the third cutoff value can be from 500 kDa to 550 kDa, 550 kDa to 600 kDa, 600 kDa to 650 kDa, 650 kDa to 700 kDa, or 700 kDa to 750 kDa.

In some embodiments, the third cutoff value can be from 750 kDa to 50 nm. In some embodiments, the third cutoff value can be from 750 kDa to 800 kDa, 800 kDa to 850 kDa, 850 kDa to 900 kDa, 900 kDa to 950 kDa, 950 kDa to 1000 kDa, 1000 kDa to 25 nm, or 25 nm to 50 nm.

In some embodiments, the third cutoff value can be from 50 nm to 0.2 μm. In some embodiments, the third cutoff value can be from 50 nm to 100 nm, 100 nm to 150 nm, or 150 nm to 0.2 μm.

In some embodiments, the polymerized hemoglobin can be prepared by a process that can further include filtering the fourth retentate fraction comprising the polymerized hemoglobin by ultrafiltration against a fifth filtration membrane, thereby forming a fifth retentate fraction comprising the polymerized hemoglobin with a molecular weight above a fourth cutoff value and a fifth permeate fraction comprising species having a molecular weight below the fourth cutoff value and above the third cutoff value.

In some embodiments, the fourth cutoff value can be from the third cutoff value to 0.2 μm.

In some embodiments, the fourth cutoff value can be from the third cutoff value to 500 kDa. In some embodiments, the fourth cutoff value can be from the third cutoff value to 350 kDa, 350 kDa to 400 kDa, 400 kDa to 450 kDa, or 450 kDa to 500 kDa.

In some embodiments, the fourth cutoff value can be from 500 kDa to 750 kDa. In some embodiments, the fourth cutoff value can be from 500 kDa to 550 kDa, 550 kDa to 600 kDa, 600 kDa to 650 kDa, 650 kDa to 700 kDa, or 700 kDa to 750 kDa.

In some embodiments, the fourth cutoff value can be from 750 kDa to 50 nm. In some embodiments, the fourth cutoff value can be from 750 kDa to 800 kDa, 800 kDa to 850 kDa, 850 kDa to 900 kDa, 900 kDa to 950 kDa, 950 kDa to 1000 kDa, 1000 kDa to 25 nm, or 25 nm to 50 nm.

In some embodiments, the fourth cutoff value can be from 50 nm to 0.2 μm. In some embodiments, the fourth cutoff value can be from 50 nm to 100 nm, 100 nm to 150 nm, or 150 nm to 0.2 μm.

In some embodiments, the filtration membrane can be rated for retaining solutes having a molecular weight greater than 0.2 μm, thereby forming a retentate fraction including species having a molecular weight of greater than 0.2 μm and a permeate fraction including the polymerized hemoglobin having a molecular weight of less than 0.2 μm and the low molecular weight hemoglobin species.

In some embodiments, the ultrafiltration includes tangential-flow filtration. Tangential-flow filtration has a meaning as described herein.

In some embodiments, the polymerized hemoglobin can be prepared by a process that can further comprise filtering the permeate fraction including the polymerized hemoglobin and the low molecular weight hemoglobin species by ultrafiltration against a second filtration membrane, thereby forming a second retentate fraction including the polymerized hemoglobin having a molecular weight below 0.2 μm and above a cutoff value and a second permeate fraction comprising the low molecular weight hemoglobin species. In some embodiments, the polymerized hemoglobin can have a molecular weight from 50 nm to below 0.2 μm, 750 kDa to 50 nm, 500 kDa to 750 kDa, or 300 kDa to 500 kDa.

In some embodiments, the cutoff value can be from 300 kDa to 0.2 μm.

In some embodiments, the cutoff value can be from 50 nm to 0.2 μm. In some embodiments, the cutoff value can be from 50 nm to 100 nm, 100 nm to 150 nm, or 150 nm to 0.2 μm.

In some embodiments, the cutoff value is from 750 kDa to 50 nm. In some embodiments, the cutoff value can be from 750 kDa to 800 kDa, 800 kDa to 850 kDa, 850 kDa to 900 kDa, 900 kDa to 950 kDa, 950 kDa to 1000 kDa, 1000 kDa to 25 nm, or 25 nm to 50 nm.

In some embodiments, the cutoff value is from 500 kDa to 750 kDa. In some embodiments, the cutoff value can be from 500 kDa to 550 kDa, 550 kDa to 600 kDa, 600 kDa to 650 kDa, 650 kDa to 700 kDa, or 700 kDa to 750 kDa.

In some embodiments, the cutoff value is from 300 kDa to 500 kDa. In some embodiments, the cutoff value can be from 300 kDa to 350 kDa, 350 kDa to 400 kDa, 400 kDa to 450 kDa, or 450 kDa to 500 kDa.

In some embodiments, the perfusion solution can comprise from 1% by weight to 5% by weight albumin, based on total weight of the perfusion solution.

In some embodiments, the perfusion solution can have an osmolarity from 270 to 370 mOsm. In further embodiments, the perfusion solution can have an osmolarity from 270 to 290 mOsm, 290 to 310 mOsm, 310 to 330 mOsm, 330 to 350 mOsm, or 350 to 370 mOsm.

In some embodiments, the perfusion solution can have a viscosity from 2 cP to 4.5 cP at normothermic conditions. In further embodiments, the perfusion solution can have a viscosity from 2 cP to 3 cP, 3 cP to 4 cP, or 4 cP to 4.5 cP.

In some embodiments, the perfusion solution can have a viscosity from 2.9 to 3.7 cP at normothermic conditions. In further embodiments, the perfusion solution can have a viscosity from 2.9 to 3.1 cP, 3.1 to 3.3 cP, 3.3 to 3.5 cP, or 3.5 to 3.7 cP.

In some embodiments, the perfusion solution can have a colloid osmotic pressure from 14 mm Hg to 20 mm Hg. In further embodiments, the perfusion solution can have a colloid osmotic pressure from 14 mm Hg to 16 mm Hg, 16 mm Hg to 18 mm Hg, or 18 mm Hg to 20 mm Hg.

In some embodiments, the perfusion solution can have a colloid osmotic pressure from 16.8 mm Hg to 17.6 mm Hg.

In some embodiments, the polymerized hemoglobin can be synthesized using a molar ratio from 20:1 to 40:1 of glutaraldehyde to hemoglobin. In some embodiments, the polymerized hemoglobin can be synthesized using a molar ratio from 25:1 to 35:1 of glutaraldehyde to hemoglobin. In further embodiments, the ratio can be at least 1:1 (e.g., at least 2:1, at least 5:1, at least 10:1, at least 20:1, at least 30:1, at least 40:1, at least 50:1, at least 60:1, at least 70:1, at least 80:1, or at least 90:1). In certain embodiments, the ratio can be 100:1 or less (e.g., 90:1 or less, 80:1 or less, 70:1 or less, 60:1 or less, 50:1 or less, 40:1 or less, 30:1 or less, 20:1 or less, or 10:1 or less).

The range can vary between any of the minimum values described above to any of the maximum values described above. For example, the polymerized hemoglobin can be synthesized at a molar ratio that can range from 1:1 to 100:1 (e.g., from 1:1 to 10:1, 10:1 to 20:1, 20:1 to 30:1, 30:1 to 40:1, 40:1 to 50:1, 50:1 to 60:1, 60:1 to 70:1, 70:1 to 80:1, 80:1 to 90:1, 90:1 to 100:1).

In some embodiments, the partial pressure of oxygen at which 50% of the polymerized hemoglobin is saturated with oxygen can be from 1 mm Hg to 50 mm Hg. In further embodiments, the partial pressure of oxygen at which 50% of the polymerized hemoglobin is saturated with oxygen can be from 10 mm Hg to 12 mm Hg, 12 mm Hg to 14 mm Hg, 14 mm Hg to 16 mm Hg, 16 mm Hg to 18 mm Hg, or 18 mm Hg to 20 mm Hg. In some embodiments, the partial pressure of oxygen at which 50% of the polymerized hemoglobin is saturated with oxygen can be from 14 mm Hg to 16 mm Hg. As used herein, saturation with oxygen refers to the binding of heme in hemoglobin with oxygen molecules. The degree of oxygen saturation of hemoglobin is dependent on the number of heme units that are bound to oxygen (e.g., 50% saturation with oxygen means that half of the heme units are bound to oxygen molecules).

In some embodiments, the polymerized hemoglobin can exhibit an auto-oxidation rate at 37° C. of from 0.0020 to 0.0085 h⁻¹. In further embodiments, the polymerized hemoglobin can exhibit an oxidation rate of from 0.0020 to 0.0045 h⁻¹, 0.0045 to 0.0065 h⁻¹, or 0.0065 to 0.0085 h⁻¹. In some embodiments, the polymerized hemoglobin can exhibit an oxidation rate of from 0.0045 to 0.0065 h⁻¹.

In some embodiments, the perfusion solution can further include one or more of a metabolic suppressant agent. As used herein, “metabolic suppressant agent” refers to an agent used to suppress the metabolic demand that is required to keep organs and/or tissue at normothermic conditions during preservation for transplantation.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures and other reaction ranges and conditions that can be used to optimize the product purity and yield obtained from the described process. Only reasonable and routine experimentation will be required to optimize such process conditions.

### Example 1. Polymerized Human Hemoglobin Based Oxygen Carrier for Maintaining Lung Viability During Normothermic Ex Vivo Lung Perfusion (NEVLP)

### Overview

Normothermic ex vivo lung perfusion (NEVLP) is able to resuscitate marginal lung allografts to make more organs available for transplantation. During normothermic perfusion, cellular metabolism is active, creating a need for an oxygen (O₂) carrier to adequately oxygenate the graft. As an O₂ carrier, red blood cells (RBCs) are prone to hemolysis in perfusion circuits leading to cell-free hemoglobin (Hb) toxicity, oxidative tissue damage, and cell death. Polymerized human Hb (PolyhHb) is a class of Hb-based O₂ carrier (HBOC) that can be synthesized and purified at a large scale such that it can transport and offload O₂ to support cellular metabolism, while not demonstrating cytotoxic side-effects. In this example, a next-generation PolyhHb was synthesized and purified at the 30 L pilot scale with a suite of analytical techniques including size exclusion chromatography high pressure liquid chromatography (SEC-HPLC) to validate that the majority of low molecular weight Hb polymers (<500 kDa) were removed from the solution. The PolyhHb was then added to an existing colloid solution and compared to both RBC and asanguinous perfusates in a rat NEVLP model. The pulmonary artery pressure and pulmonary vascular resistance were both higher in lungs perfused with RBCs, likely due to vasoconstriction from hemolysis and subsequent exposure to cell-free Hb. Lungs perfused with PolyhHb also demonstrated greater oxygenation than those perfused with RBCs and elicited less cellular damage and edema than both other perfusates.

### Background

Normothermic machine perfusion (NMP) has shown promise in the field of organ storage and preservation to improve the viability of transplanted organs. Studies have shown that NMP can resuscitate ECD organs to obtain pre-transplant quality comparable to nonmarginal organs for subsequent transplantation. There is a significant metabolic demand maintaining organs at normothermia and this metabolic demand is only exacerbated in DCD organs. There is a demand for an artificial RBC substitute that can store and transport oxygen (O₂) to meet the metabolic demands of perfused organs while not being subject to hemolysis or Hb/heme/iron toxicity.

Of all the different types of HBOCs being studied to carry and offload O₂ to surrounding tissue, polymerized Hb (PolyHb) has the ability to be synthesized and purified at large scale. Any adverse side-effects are the result of the presence of cell-free Hb and low molecular weight (MW) Hb polymers (<500 kDa) that extravasate out of the circulation into the tissue space, which leads to nitric oxide (NO) scavenging and subsequent vasoconstriction, systemic hypertension, and oxidative tissue injury. Elimination of these low MW Hb species from the NMP perfusate may mitigate these deleterious side-effects. In ex vivo lung perfusion (EVLP), there is heightened concern about the development of edema which further necessitates the need for development of a perfusate that will not damage the especially delicate tissues of the lung. Currently, the majority of EVLPs, such as those that follow the Toronto Protocol, contain no O₂ carrying molecule in the perfusate. Without an O₂ carrier in the perfusate, the metabolic demand during normothermic EVLP (NEVLP), especially when perfusing DCD lungs, is not fully met.

This example utilizes a clinically relevant, validated, lung DCD model and NEVLP platform to assess the ability of a polymerized human Hb (hHb) (PolyhHb) HBOC perfusate to meet DCD organ metabolic demands and evaluate organ quality as compared to an RBC and asanguinous perfusate.

### Materials and Methods

Materials. Glutaraldehyde (70/6), sodium chloride (NaCl), potassium chloride (KCl), sodium hydroxide (NaOH), sodium dithionite (Na₂S₂O₄), calcium chloride (CaCl₂·2H₂O), sodium lactate, Nacetyl-L-cysteine (NALC), sodium cyanoborohydride (NaCNBH₃), sodium phosphate dibasic (Na₂HPO₄), sodium phosphate monobasic (NaH₂PO₄), trichloroacetic acid (C₂HCl₃O₂), sodium acetate (C₂H₃NaO₂), ascorbic acid (C₆HO₆), and citrate buffer were purchased from Sigma-Aldrich (St. Louis, MO). Hollow fiber tangential flow filtration (TFF) modules (polyethersulfone (PES) 0.2 μm and polysulfone (PS) 500 kDa) were purchased from Spectrum Laboratories (Rancho Dominguez, CA). All other chemicals were purchased from Fisher Scientific (Pittsburgh, PA). Expired human RBC units were generously donated by Canadian Blood Services, Ottawa, Canada.

William's E Medium (A12176-01) was purchased from Gibco, Thermo Fisher Scientific (Waltham, MA). Sprague-Dawley rat RBCs were purchased from Innovative Research Inc. (Novi, MT). Male Sprague-Dawley rats were purchased from Envigo (Indianapolis, IN) and housed under pathogen-free conditions at The Ohio State University Animal Facility. All procedures were humanely performed according to the NIH and the National Research Council's Guide for the Humane Care and Use of Laboratory Animals and with the approval of The Ohio State University Institutional Animal Care and Use Committee (IACUC Protocol #2012A00000135-R2)

PolyhHb Synthesis. The PolyhHb was produced from a single pilot batch. Twenty units of expired human RBCs were added to 7 L of 0.9 wt % saline to achieve a pooled hematocrit of 22%. RBCs were washed with 6 volume difiltrations of saline over a 0.65 μm modified polyethersulfone (mPES) TFF filter. The RBC solution was then concentrated down to 10 L before being lysed for 1 hour with an equal volume of phosphate buffer (PB) (3.75 mM, pH 7.4). Cell debris was eliminated by filtration of the RBC lysate through a 500 kDa PES filter with purified Hb and other intracellular proteins present in the permeate flowing into a 30 L bioreactor. 470 g of Hb was allowed to flow into the bioreactor before the permeate feed line was closed. The Hb in the bioreactor had deionized water, NaCl, and KCl added to it to bring the final solution to 18.2 g_{Hb}/L in phosphate buffered saline (PBS). The bioreactor was held under a nitrogen (N₂) head at 14° C. overnight.

The next morning, the cooling coils were turned off and the bioreactor was brought to and maintained at 37° C. using a heating jacket. The Hb solution was deoxygenated using an external flow loop with a G420 X40 gas-liquid exchanger (3M, Maplewood, MN) and N₂ as the sweep gas. Once the partial pressure of O₂ (pO₂) reached below 5 mm Hg on a Rapidlab 248 (Siemens, Malvern, PA) blood gas analyzer (BGA), a bolus of 6 g of Na₂S₂O₄ was added to reduce the pO₂ to below readable levels on the BGA. Upon full deoxygenation of the solution, a 0.75 wt %/o glutaraldehyde solution in PBS (0.1 M, pH 7.4) was added in a 30:1 molar ratio glutaraldehyde to hemoglobin over a period of 3 hours. The solution was allowed to mix for an additional hour after glutaraldehyde addition was completed. After polymerization was completed, the heating jacket was removed and the cooling coils were turned back on. NaCNBH₃ was used to quench the reaction at a 7:1 molar ratio of NaCNBH₃ to glutaraldehyde through rapid delivery of a 3.5 wt % solution of NaCNBH₃ in PBS (0.1 M, pH 7.4). The solution continued to quench and cool overnight.

PolyhHb purification followed methods described previously in the literature. The sole deviation was that the number of diafiltrations was increased from 8 to 16 to ensure the final PolyhHb product contained <5% low MW Hb species (<500 kDa). The final product was concentrated to 11.5 g/dL and stored at −80° C.

Protein Quantification. The total Hb concentration and percentage of methemoglobin (metHb) were determined using the cyanomethemoglobin method. This assay was used to quantify PolyhHb during production, after purification, and during NEVLP. Cell-free Hb in RBC perfusates was quantified by centrifuging the perfusate until the RBCs formed a pellet. The resulting supernatant was then analyzed via the cyanomethemoglobin method. To determine the total Hb concentration in RBCs, RBCs were lysed through freeze thaw cycles followed by dilution in PB (3.75 mM, pH 7.4). The lysed RBCs were again centrifuged down to a pellet and the supernatant assayed for the total Hb concentration.

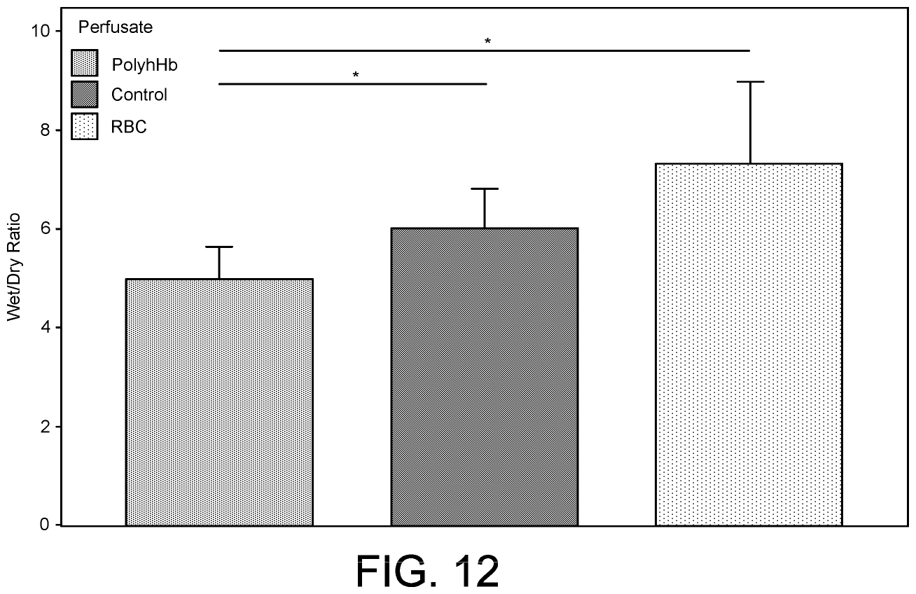

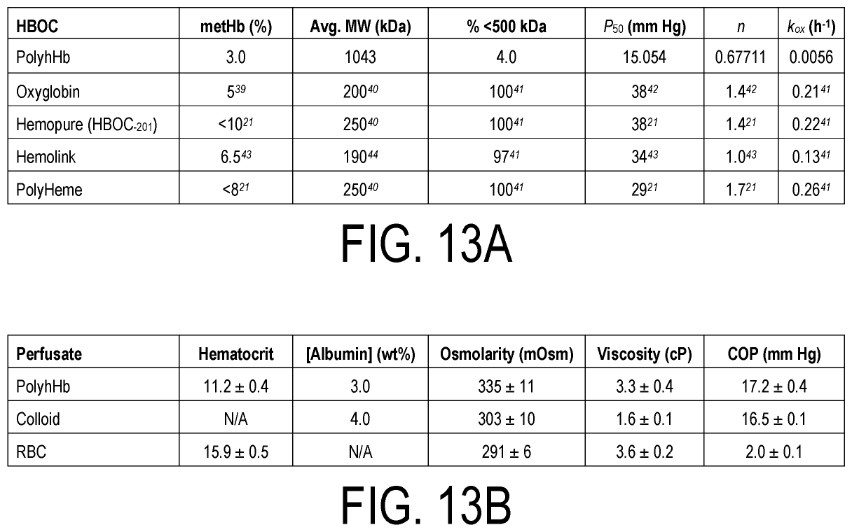

Biophysical Parameters. The oxygen equilibrium curve, MW distribution, and auto-oxidation kinetics for PolyhHb and Hb were all measured as previously described in the literature. Briefly, oxygen equilibrium curves were measured using a Hemox Analyzer (TCS Scientific, New Hope, PA) at 37° C. and pH 7.4. The MW distribution was estimated by performing size exclusion high pressure liquid chromatography (SEC-HPLC) using an Acclaim SEC-1000 column (Thermo Scientific, Waltham, MA) on a Thermo Scientific UHPLC System using MW standards. Auto-oxidation kinetics were measured via UV-visible spectrometry over 24 hours at 37° C. The solution viscosity was measured using a DV3T-CP cone and plate viscometer (Brookfield AMETEK, Middleboro, MA), and osmolarity was measured using a Gonotech 010 freezing point osmometer (Gonotech GmbH, Berlin, Germany). Colloid osmotic pressure (COP) was measured using a Wescor 4420 Colloid Osmometer (Wescor, Logan, UT). FIG. 13A lists the biophysical properties of PolyhHb and prior generations of HBOCs.

Perfusate Formulation. Perfusates were formulated using William's cell culture media as the primary fluid at a final volume of 165 mL. For the HBOC perfusate, PolyhHb was diluted with William's media to a final concentration of 3.7±0.1 g/dL. Twenty-five percent human serum albumin (HSA) was added such that the final perfusate consisted of 3% HSA by weight. Rat RBCs were diluted to a final hematocrit of 15% to be in line with the Lund protocol. The asanguinous control consisted of 4% HSA in William's media. Each of these perfusates was brought to 37° C. and pH 7.4 using THAM buffer before beginning NEVLP. FIG. 13B lists the composition of the NEVLP perfusates.

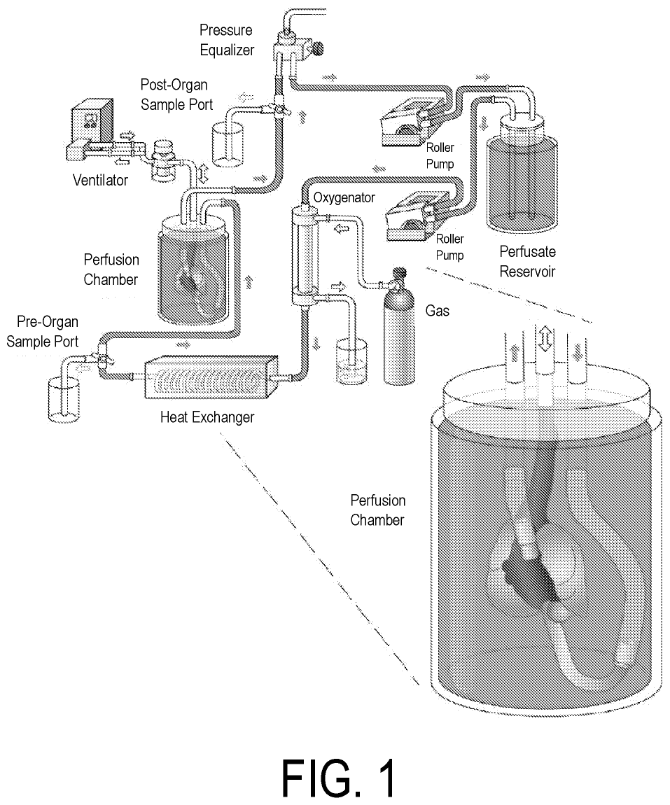

NEVLP. Briefly, rats were fully anesthetized by a ketamine and xylazine injection via intraperitoneal injection then shaved and positioned for lung procurement. The incision was started by cutting the abdomen to open the peritoneum. Heparin (100 IU/kg) was injected via the inferior vena cava (IVC) and allowed to circulate for 10 minutes. Rats were then connected to the ventilator via tracheostomy and exsanguinated by cutting the IVC. The thoracic cavity was opened, and the pulmonary vein and left atrium were cannulated via transapical approach. The heart-lung bloc was removed from the chest cavity with the lung-no-touch technique before being connected to the ex vivo perfusion system as shown in FIG. 1.

Lungs were ventilated for 2 hours or until pulmonary artery pressure (PA pressure) exceeded 100 mmHg. Ventilation was performed with ambient air at 60 bpm and tidal volume of 4 mL/kg of rat with a positive end expiratory pressure (PEEP) of 2 cmH₂O. The perfusion flow rate was set at 20% of estimated cardiac output (75 mL/kg of rat). Perfusate samples were collected at each timepoint of NEVLP and snap frozen until needed for analysis.

Post-NEVLP Analysis. Lactate dehydrogenase (LDH) released into the perfusate was measured using a LDH cytotoxicity detection kit (Clontech Laboratories, Mountain View, CA) and following the manufacturer's instructions. The right inferior lobe was used for wet to dry ratio determination. The lobe was weighed immediately upon perfusion termination for wet weight, dried at 60° C. for 48 hr., and then weighed again for the dry weight.

Lung Histology. Snap frozen superior lung lobe samples were homogenized in 10 volume equivalents of deionized water. 1 mL of the homogentate was incubated (60 min, 50° C.) with 500 μL of a solution containing 1 M hydrochloric acid (HCl) and 10% trichloratic acid. Samples were then centrifuged at 8,000 g for 15 minutes and 375 μL of supernatant was mixed with 125 μL of a 2% ascorbic acid solution to reduce ferric iron. To quantify, ferrous iron 100 μL of a solution containing 1 g/L of FerroZine™ (ACROS Orgnaics, Geel, Belgium) and 1.5 M sodium acetate was added to the aliquot. Samples were allowed to develop for 30 minutes, after which the absorbance at 562 nm was measured using a BioTek Synergy HTX plate reader.

Paraffin embedded tissue blocks from the lung middle lobe were prepared, and tissue was sectioned (5-micron thickness) by the University of Maryland Baltimore Histology Core. Sections were dewaxed and hydrated in graded ethanol percentages. Heat-mediated antigen retrieval was performed using pH 6.0 citrate buffer. The solution was brought to a boil, and then allowed to cool for 30 minutes. Once cool, sections were incubated at room temperature for 30 minutes with 3% horse serum. Sections were then incubated overnight at 4° C. with an antibody against the Hb α-chain (1:300, Abcam, Ab92492). A biotinylated secondary antibody was used for Hb α-chain staining with sections incubated for 30 minutes (1:300, Invitrogen, 31820). Sections were then incubated for 30 minutes with avidin and biotinylated horseradish peroxidase (VECTASTAIN Elite® ABC Kit, Vector Laboratories), and incubated for 3 minutes with 3,3′-diaminobenzidine and H₂O₂ (SIGMAFAST™, Sigma). Sections were then counterstained in hematoxylin (Gil no. 2, Fisher). All images were obtained using a Leica DM4-B TL bright field microscope (Leica, Wetzlar, Germany) and captured with LAS X software at 630 times total magnification using a 63 times objective with oil immersion at 22.02 milliseconds of exposure.

Statistical Analysis. All statistical analysis was done using RStudio (Version 1.4.1106, RStudio, Inc., Boston, MA) using a one-way ANOVA to determine significance. Significance was reported to an a value of 0.05. In all figures, an asterisk is used to indicate significance between groups. A pound sign is used to indicate significance within a group compared to its initial value.

### Results and Discussion

PolyhHb Biophysical Properties. The potential ability of the PolyhHb synthesized in this study to function as a perfusate can be assessed by a variety of in vitro parameters. The metHb level (%), average MW, pO₂ at which 50% of the Hb or PolyhHb is saturated with O₂ (P₅₀), cooperativity coefficient (n), percentage of low MW species in solution (<500 kDa), and auto-oxidation rate constant (kₒₓ) are listed in FIG. 13A for both the PolyhHb produced in this example as well as for prior generations of commercial HBOCs.

The percentage of metHb in the PolyhHb produced in this study is marginally lower than other high-MW PolyhHbs. This can likely be explained by the significantly lower kₒₓ compared to previous generations of commercial HBOCs. Hemolink (Hemosol Inc.), Hemopure (Biopure Corp.), Oxyglobin (Biopure Corp.), and PolyHeme (Northfield Laboratories) are all commercial polymerized Hbs that failed Phase III clinical trials and each of these HBOCs were reported to have a kₒₓ between 0.13-0.26 h⁻¹ which represents an auto-oxidation rate 20-40 times faster than the PolyhHb produced in this example. The lower rate of auto-oxidation means that in an NEVLP circuit, the PolyhHb HBOC formulation will retain its ability to load and offload O₂ as intended over a longer period of time, since most of the Hb will exist in the ferrous form (HbFe²⁺) instead of the oxidized ferric form (HbFe³⁺, metHb) which cannot bind O₂. This is invaluable in NEVLP experiments because prior HBOCs can lose a third of their O₂-carrying capacity in as little as an hour during NMP.

The average MW of the pilot scale PolyhHb preparation is 5 times larger than prior generations of commercial HBOCs. Additionally, the PolyhHb produced in this example only contains a small fraction of the low MW species (<500 kDa) compared to previous generation of commercial HBOCs. As mentioned above, the high concentrations of Hb and low MW Hb polymers in prior generations of commercial HBOCs have created a host of disqualifying problems preventing them from being successful in transfusion medicine despite their ability to carry and offload O₂ as intended.

The P₅₀ and n are lower for this PolyhHb compared to previous generations of commercial HBOCs, which were synthesized in the low O₂-affinity tense quaternary state (T-state). These prior HBOCs readily offloaded O₂ to surrounding tissue to support metabolic activity. The indiscriminate offloading of O₂ can actually be detrimental to the organ due to a phenomenon called autoregulation, whereby in the presence of high O₂ levels, the organism limits O₂ consumption in an attempt to keep tissue O₂ levels constant. Additionally, too much O₂ offloading during perfusion has been shown to generate reactive oxygen species (ROS) which are highly detrimental to graft survivability. Therefore, it is more beneficial to design an HBOC with an O₂ affinity similar to or less than that of RBCs in order to support metabolic activity without inducing autoregulation or ROS formation. Hence, the P₅₀ of the PolyhHb described in this study being half that of previous generations of commercial HBOCs may actually prove to be beneficial given that the P₅₀ of human RBCs is about 26 mmHg.

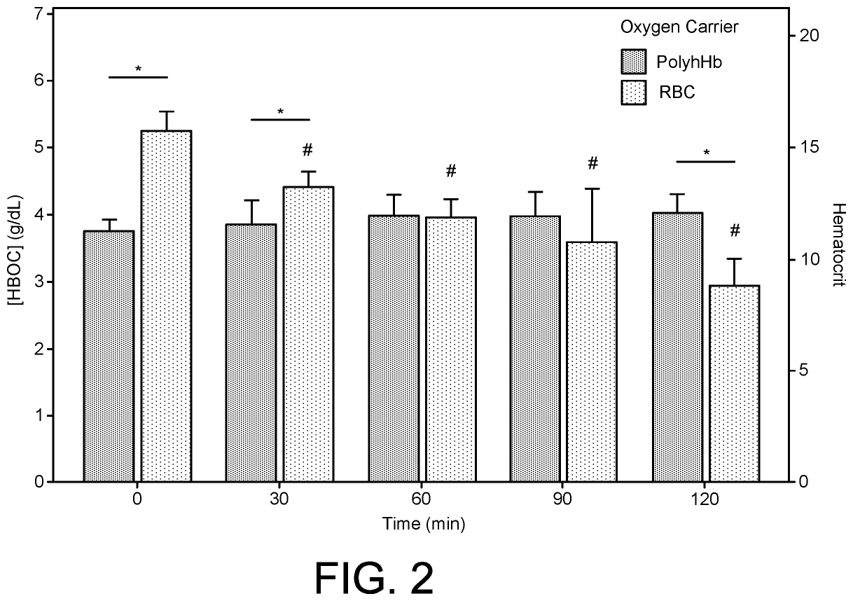

NEVLP. In order for the full potential of NEVLP to be realized, an Oz carrier needs to be present in the perfusate in order to support the metabolic activity of the lung during NMP. The PolyhHb perfusate concentration remains very stable throughout the perfusion (FIG. 2), which indicates no extravasation of the material into the tissue space of the lung. There is an insignificant increase in concentration due to evaporative concentration of the PolyhHb in the NEVLP circuit; however, no point is significantly different compared to the initiation of NEVLP.

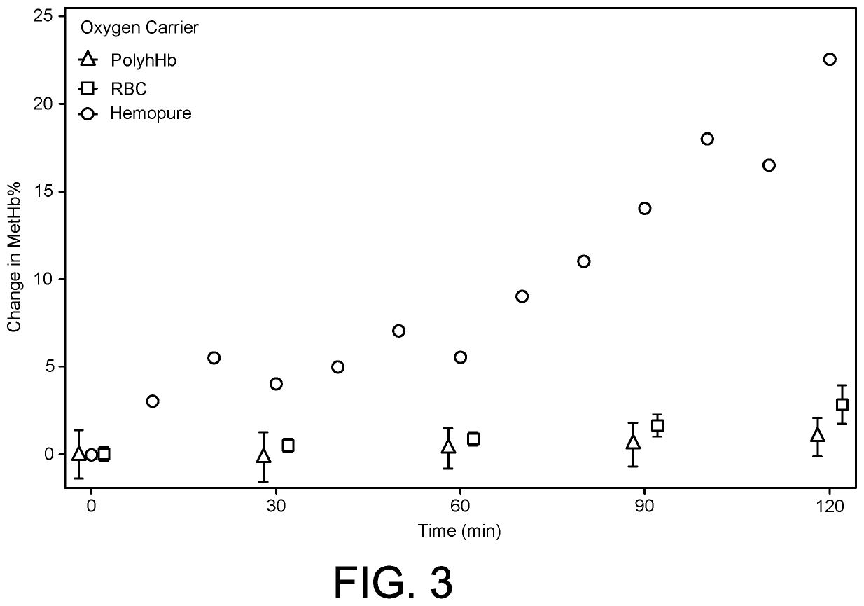

As shown in FIG. 13A, the PolyhHb synthesized in this work exhibited exceptionally lower kₒₓ compared to any previously synthesized polymerized Hb. This holds true beyond in vitro experiments as demonstrated in FIG. 3. The PolyhHb perfusate demonstrated stability throughout the course of NEVLP, increasing the percentage of metHb by less than 2%. Compared to previously published HBOC perfusate data, shown in FIG. 3, which demonstrated an almost 25% increase in metHb in the perfusate. In this previous study, the group administered multiple bolus injections of vitamin C and glutathione in an effort to reduce the metHb being formed, but even with these interventions, almost a third of the final HBOC-containing perfusate was in the non-O₂ carrying ferric state (i.e. metHb) after just two hours of NMP. The rapid oxidation of Hb is ameliorated by the PolyhHb synthesized in this study, which facilitates the possibility of extended length perfusions that have not previously been viable while also reducing the rate of ROS formation by minimizing the fraction of Hb in the ferric state.

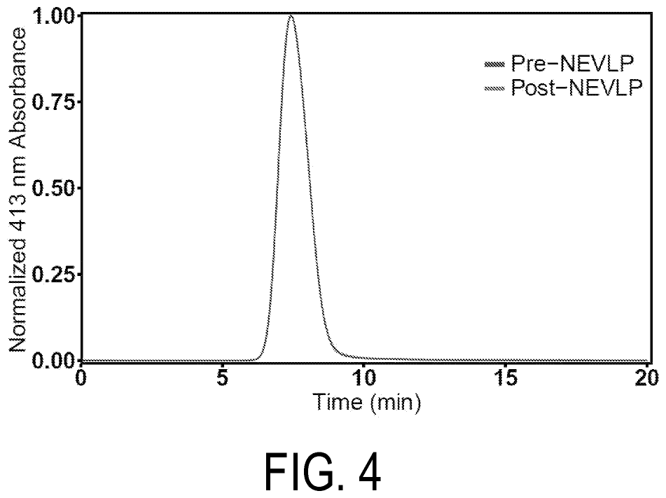

Another shortcoming of previous generations of commercial HBOCs is their propensity for low molecular weight Hb species (<500 kDa) extravasation out of circulation into the tissue space, which has detrimental side-effects that were previously elaborated on. To validate that the PolyhHb is not extravasating out of the NEVLP circuit into the lung, SEC-HPLC was run on the PolyhHb perfusate both before and after NEVLPs. These results are shown in FIG. 4 and the pre- and post-NEVLP curves overlap perfectly. This proves that PolyhHb does not extravasate into the lung tissue space during NEVLP thus increasing the safety and efficacy of this next-generation PolyhHb synthesized in this current example compared to prior commercial HBOCs. PolyhHb lack of extravasation is also verified by measuring the PolyhHb concentration during perfusion in FIG. 2, which does not change.

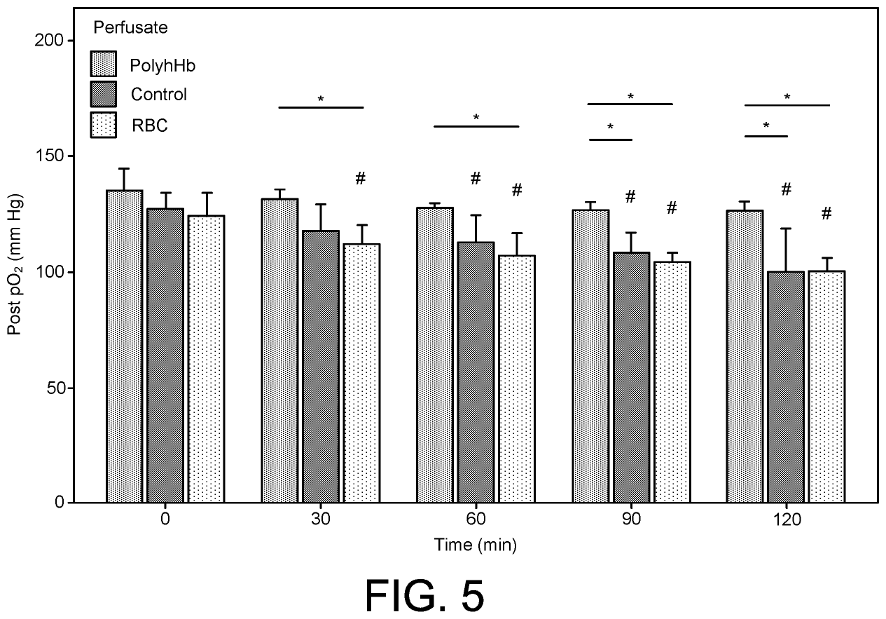

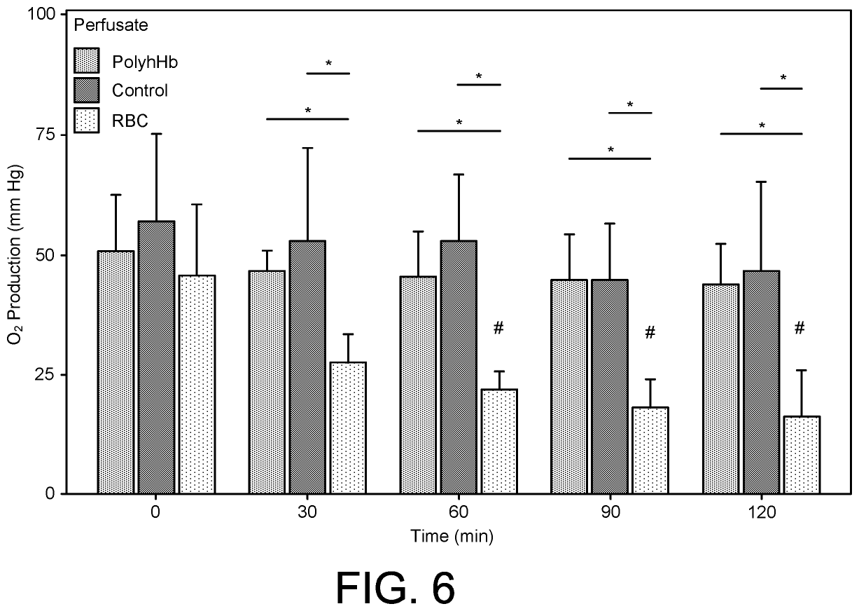

The organ metrics measured during NEVLP demonstrated equal or superior graft performance in lungs perfused with PolyhHb compared to both the asanguinous control as well as the RBC perfusate. The pO₂ of the post-bloc perfusate demonstrates the graft's ability to supply O₂ to the system and eventually potentially to circulating blood in a transplant recipient. As such, maintaining an adequate post-bloc pO₂ is an important indicator of graft health. FIG. 5 indicates that the PolyhHb perfusate best preserves the amount of O₂ that the lung is able to deliver to the system. After 30 minutes of NEVLP, lungs perfused with PolyhHb were delivering significantly more O₂ than lungs perfused with RBCs. At 90 minutes of NEVLP, PolyhHb perfused lungs were also delivering more O₂ than those perfused with the colloid control. Additionally, by 60 minutes of NEVLP, lungs perfused with both RBCs and the control experienced a significant decrease in oxygenation compared to their initial values. The lungs perfused with PolyhHb did not experience a statistically significant decrease in their ability to deliver O₂. By this metric, PolyhHb was able to sustain graft health better than both other perfusates.

The O₂-carrying ability of PolyhHb was likely the driving factor for this behavior. The increase in post-bloc pO₂ for the asanguinous control compared to PolyhHb can partially be explained by the increase in oxygenation potential of the perfusate. Having an O₂ carrier in solution will inherently facilitate more O₂ to be stored and transported in the perfusate compared to only using a colloid. Given that grafts perfused with RBCs also had an O₂ carrier in the perfusate, the improvement in PolyhHb lungs over RBC lungs can be attributed to an increase in graft health. Not only did lungs perfused with PolyhHb have a higher post-bloc pO₂, but they also did not experience a significant decrease in pO₂ over time. This also can only be attributed to an increase in graft health. Taken together, the PolyhHb perfusate sustained the ability of the lungs to provide O₂ to the perfusate better than either RBCs or an asanguinous solution.

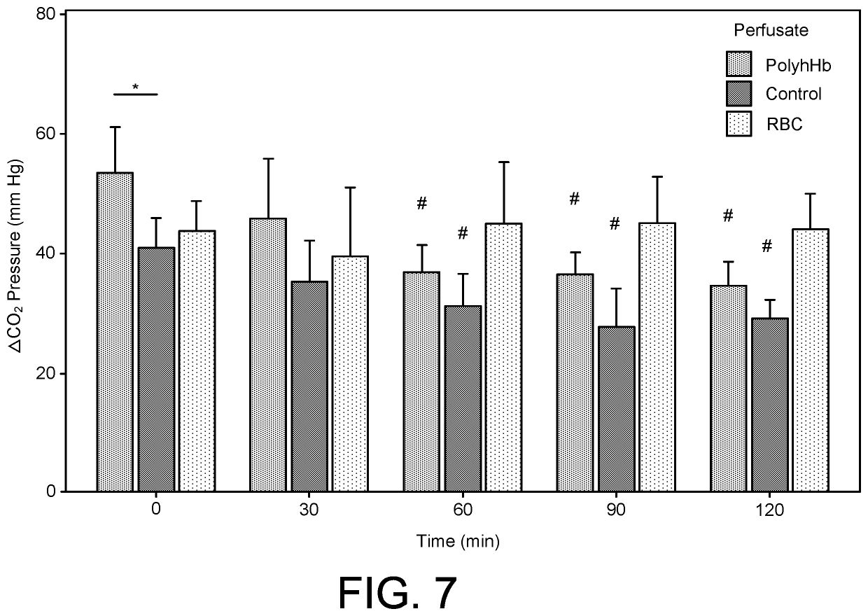

In addition to facilitating O₂ delivery to the perfusate, the other primary function of the lungs is carbon dioxide (CO₂) clearance. FIG. 7 shows the change in CO₂ tension across the heart-lung bloc as a function of time. There was no significant difference between groups after the initial time point; however, both the PolyhHb and colloid control perfusates exhibited less CO₂ clearance after 60 minutes. The RBC perfusate did not experience this decline. The colloid control was therefore the only perfusate that saw a reduction in gas exchange properties of the lungs with respect to both O₂ and CO₂.

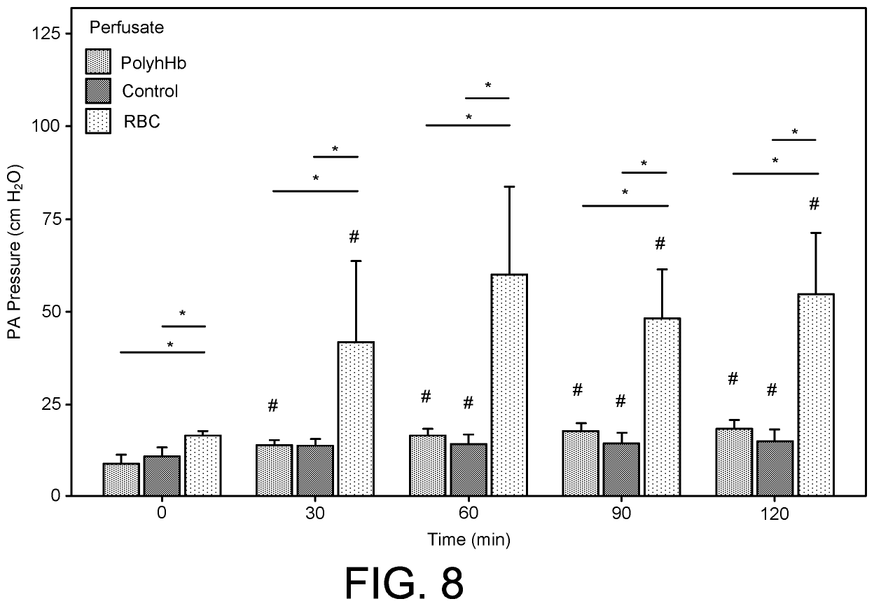

Beyond the ability to exchange gases, organ health on the circuit can be evaluated by measuring various physiological parameters during NEVLP. PA pressure is one of the most critical metrics of proper lung function, and a PA pressure elevated above 100 cm H₂O is a disqualifying event for continuation of NEVLP. All three perfusates led to an increase in PA pressure by 60 minutes as shown in FIG. 8. Lungs perfused using RBCs experienced higher PA pressures with three lungs being removed from the circuit after 60 minutes upon exceeding 100 cm H₂O. Returning to the discussion of FIG. 2, RBCs experienced significant hemolysis throughout NEVLP. Hemolysis leads to high levels of cytotoxic cell-free Hb in solution leading to, among other aforementioned side-effects, vasoconstriction. The vasoactive nature of cell-free Hb in conjunction with the fact that RBCs are lysing during NEVLP explains why the PA pressure of the RBC perfusate was much higher than the other two perfusates.

FIG. 8 shows that there is no significant difference in PA pressure between lungs perfused with PolyhHb and the asanguinous control. One of the shortcomings of previous generations of HBOCs is the presence of significant levels of low MW Hb species (<500 kDa), which elicited vasoconstriction and systemic hypertension, similar to what was observed for RBC perfusates. The lack of vasoactivity in this PolyhHb perfusate not only bodes well for the health of the organ on the circuit, but also confirms the lack of cytotoxicity due to the absence of cell-free Hb and low MW Hb species (<500 kDa) in the present PolyhHb.

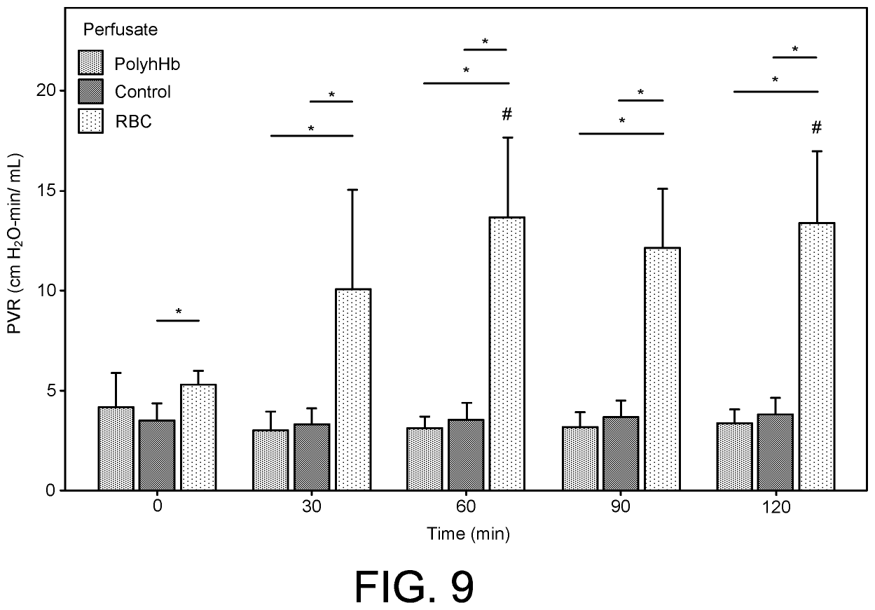

The trends displayed by the PA pressure over the course of NEVLP were mimicked by the results of tracking the pulmonary vascular resistance (PVR). FIG. 9 shows the PVR as a function of time and is similar to the results observed for the PA pressure. In both cases, the lungs perfused with RBCs exhibited prohibitively higher physiological responses compared to lungs perfused with both PolyhHb and the colloid control. Additionally, RBC perfused lungs were the only group to increase significantly from the t=0 point. These results are likely also due to the vasoactive nature of cell-free Hb derived from lysed RBCs compared to the asanguinous control or PolyhHb solution optimized to have such low MW Hb species (<500 kDa) removed from solution.

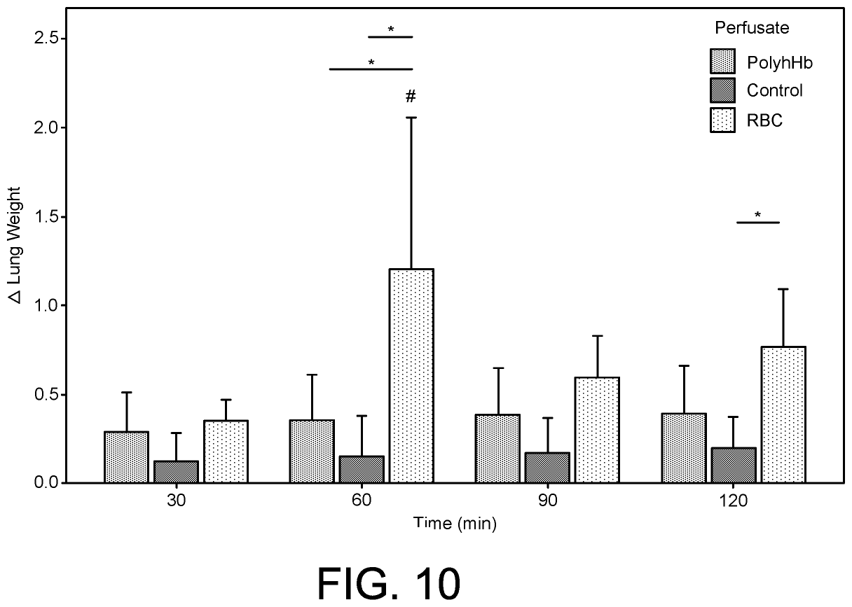

Along with vasoconstriction and systemic hypertension, the cytotoxicity caused by cell-free Hb and low MW Hb polymers (<500 kDa) is another serious side-effect associated with previous generations of PolyhHbs. On the NEVLP circuit, this manifests in real time in the change in lung weight. There are significant increases in lung weight when tissue is damaged leading to edema. The change in lung weight over time for the three perfusates is shown in FIG. 10. For RBCs, the lung weight spikes at t=60 due to the three failed lungs which were then taken off the circuit. Lungs perfused with PolyhHb or the asanguinous control did not display a significant increase in lung weight.

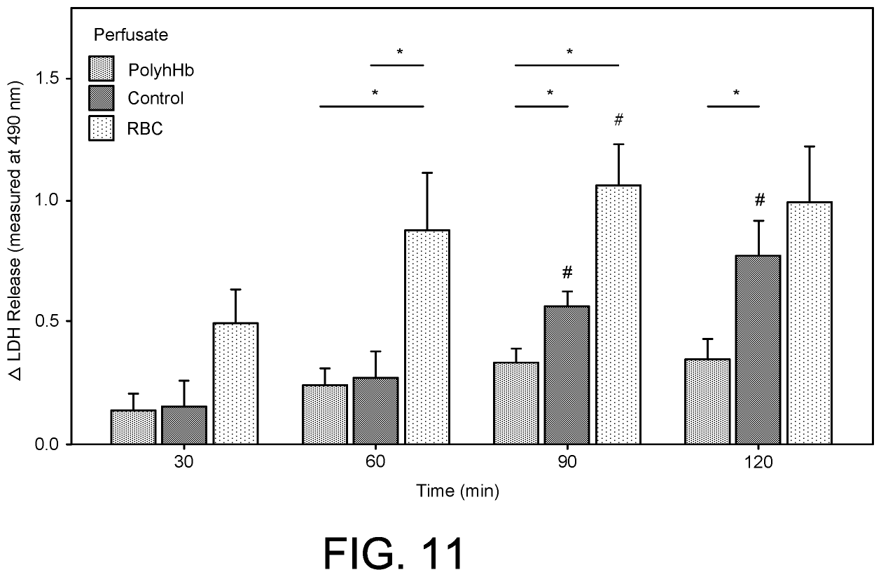

FIG. 11 shows the change in LDH during NEVLP. The change in perfusate LDH levels are lower for the PolyhHb perfusate compared to RBCs at 60 min and compared to both perfusates by 90 min. Additionally, the PolyhHb perfusate is the only group to not exhibit a significant increase in LDH compared to the t=30 min point. This means that performing NEVLPs using PolyhHb as a perfusate elicits less cellular stress and cell death compared to either RBC or asanguinous perfusates. Furthermore, the edema accumulated during NEVLP and quantified by the wet to dry ratio was shown to be significantly lower in lungs perfused with PolyhHb. The ratio of the right inferior lobe weight immediately after perfusion to the weight after 48 hours of drying is shown in FIG. 12. PolyhHb perfused lungs accumulated significantly less edema than lungs perfused with either RBCs or the asanguinous control. While the measurements made during NEVLP showed the advantages of using PolyhHb as a perfusate, the results from post-NEVLP analyses shown in FIGS. 11 and 12 show improvements in graft health by using the PolyhHb perfusate.

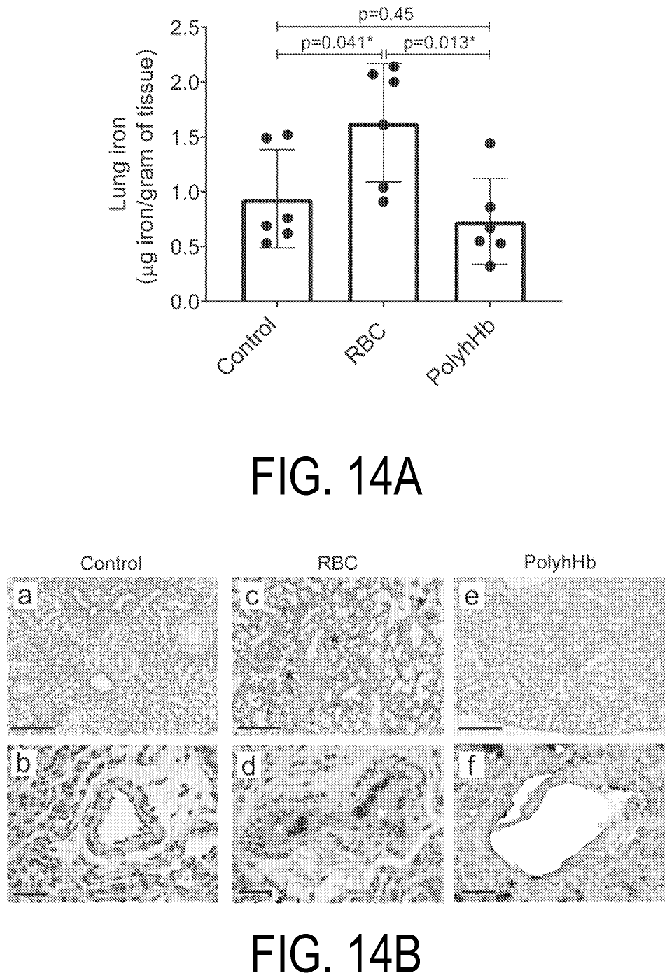

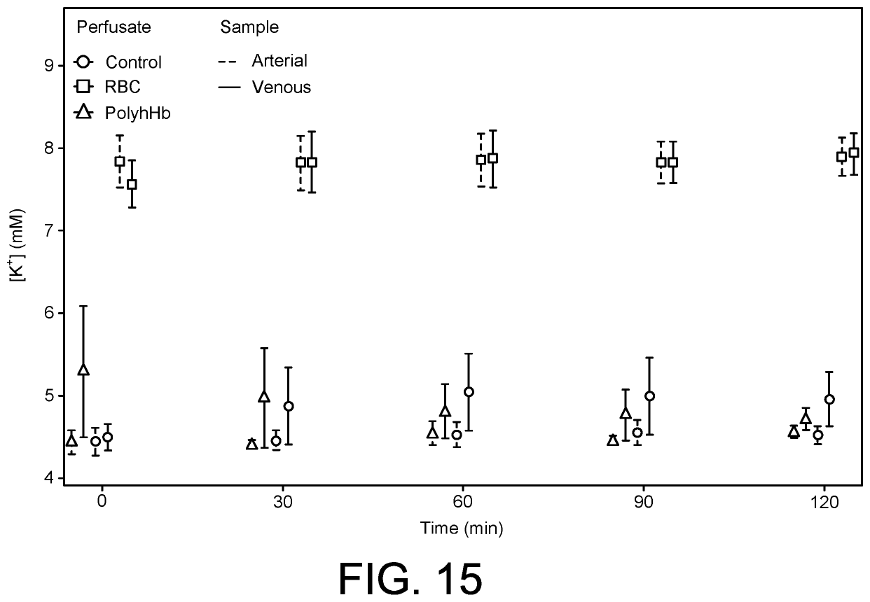

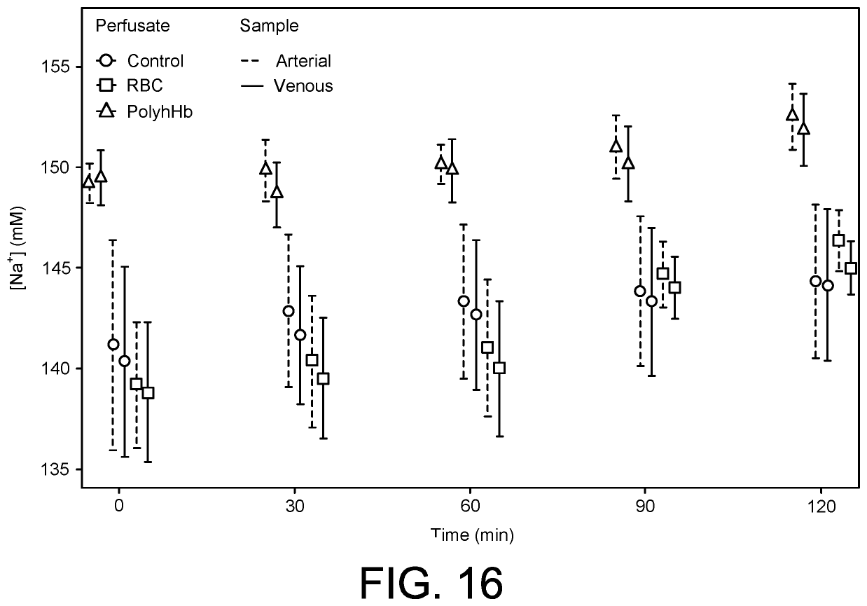

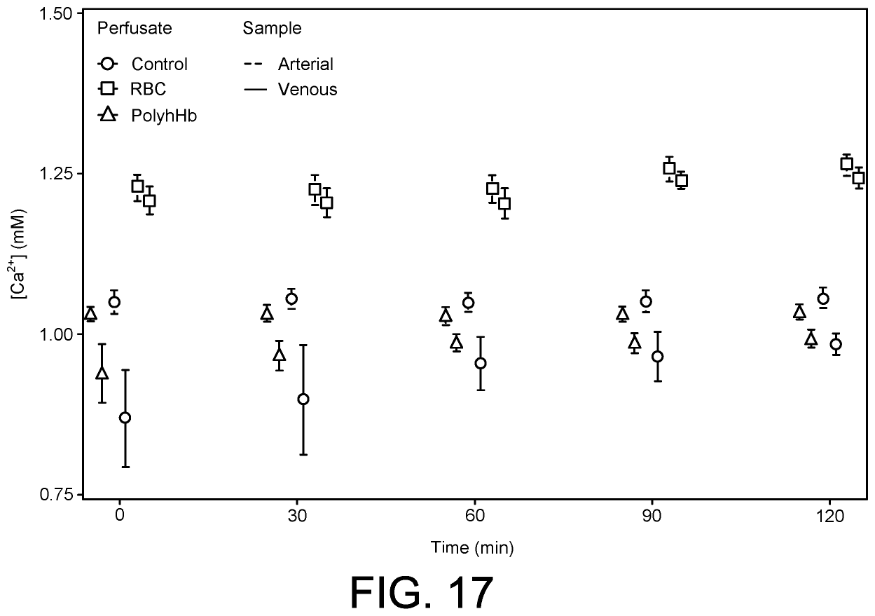

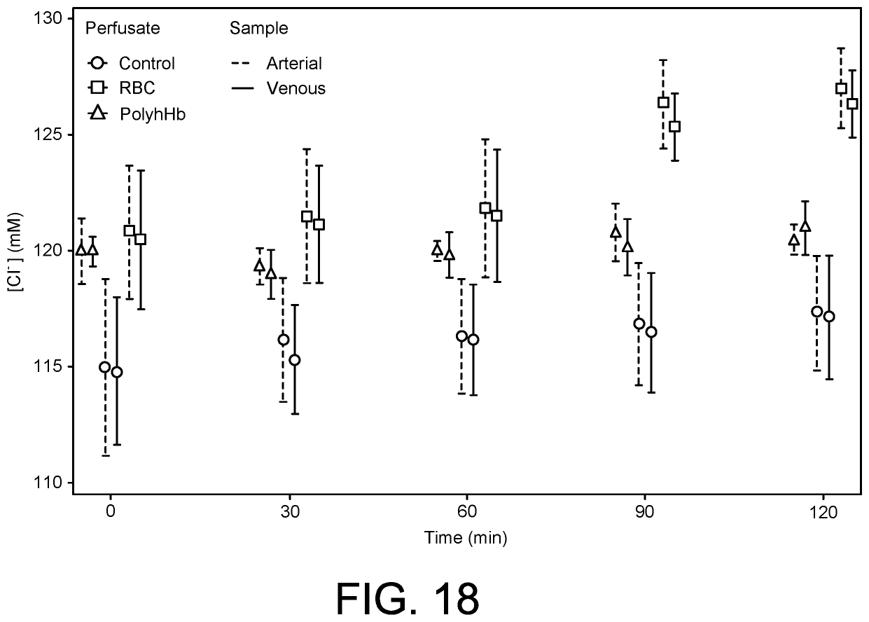

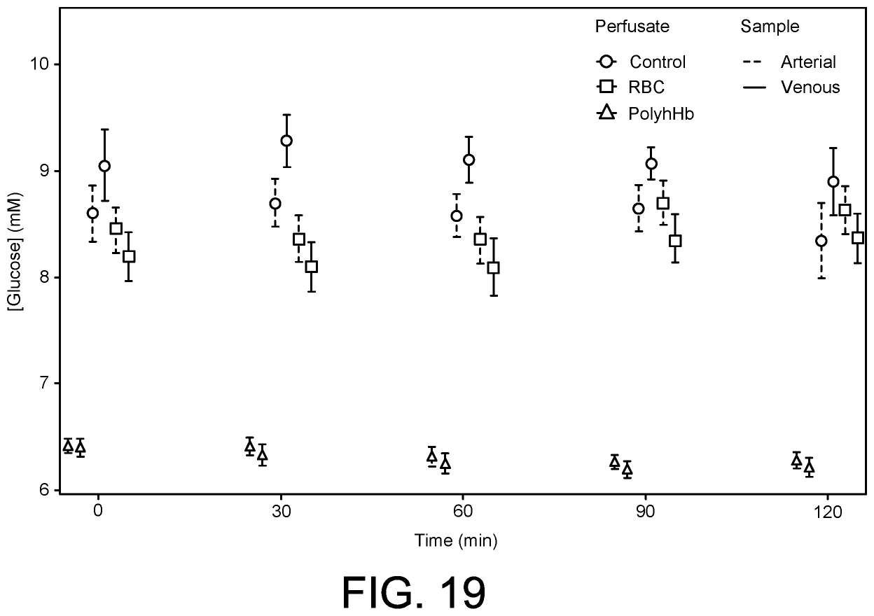

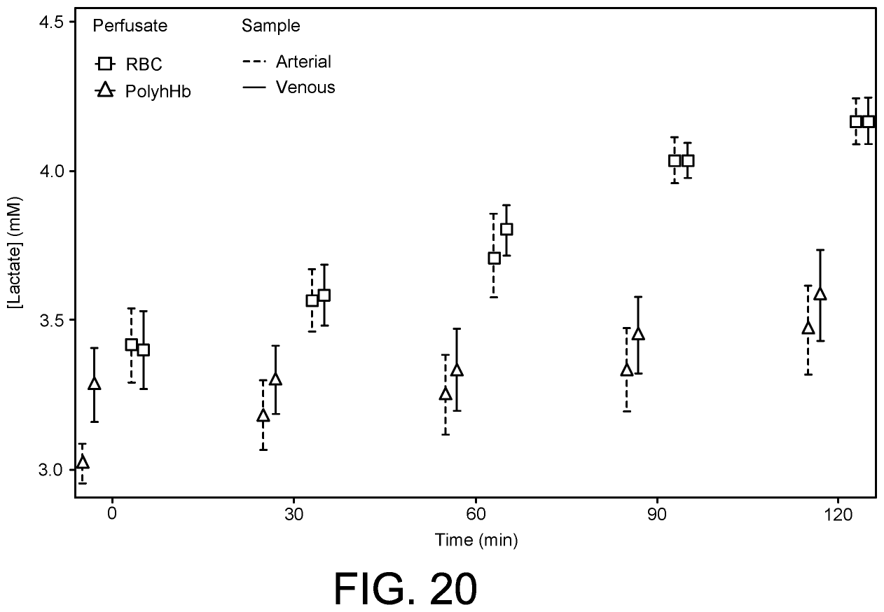

Lung tissue iron was quantified as a parameter of Hb degradation following perfusion of explanted tissue. RBC perfused tissue retained visually greater quantities of Hb compared to the control or PolyhHb perfused lungs prior to tissue homogenization and ferrozine assay analysis. Ferrozine functions as a water-soluble Fe²⁺ chelator with an absorbance at 562 nm and is therefore specific to reaction with unconjugated iron. Based on this data, RBC lung perfusion resulted in an average iron concentration equal to 1.63 μg/g of lung tissue. RBC perfused tissue contained residual iron concentrations that were 74% greater than after control perfusion and 123% greater than after PolyhHb perfusion as shown in FIG. 14A.

### Conclusion

Traditionally, NEVLP has been performed using either a colloid based asanguinous solution or an RBC-based perfusate. Both of these options have shortcomings in preserving graft health and viability during NEVLP. Earlier HBOCs including previous generations of PolyhHbs have caused detrimental side-effects due to the presence of cytotoxic cell-free Hb and other low MW Hb polymers in solution. Improvements to the synthesis and purification of PolyhHb described in this example yield a product that is significantly less likely to elicit the negative side effects observed in previous generations of PolyhHbs. Our PolyhHb demonstrates improved lung oxygenation as well as overall graft health by eliciting less edema, Hb extravasation, iron deposition, and cellular damage. This improved HBOC is a perfusate for NEVLP, which delivers O₂ while simultaneously not damaging the lungs.

### Example 2: Pilot Scale Production of Polymerized Human Hemoglobin

Polymerized human hemoglobin (PolyhHb) is being studied as a possible red blood cell (RBC) substitute for use in scenarios where blood is not available. While the O₂ carrying capacity of PolyhHb makes it appealing as an O₂ therapeutic, the commercial PolyhHb PolyHeme® (Northfield Laboratories Inc., Evanston, IL) was never approved for clinical use due to the presence of large quantities of low molecular weight polymeric (LMW) Hb species (<500 kDa), which have been shown to elicit vasoconstriction, systemic hypertension, and oxidative tissue injury in vivo. PolyhHb can by synthesized and purified using a two-stage tangential flow filtration (TFF) purification process to remove almost all undesirable Hb species (>0.2 μm and <500 kDa) in the material, in order to create a product that should be safer for transfusion. To enable future large animal studies and eventual human clinical trials, PolyhHb synthesis and purification processes need to be scaled up to the pilot scale. In this example, we describe pilot scale synthesis and purification of PolyhHb. Characterization of pilot scale PolyhHb showed that PolyhHb could be successfully produced to yield biophysical properties conducive for its use as an RBC substitute. Size exclusion chromatography showed that pilot scale PolyhHb yielded a high MW Hb polymer containing a small percentage of LMW Hb species (<500 kDa). Additionally, the auto-oxidation rate of pilot scale PolyhHb was even lower than that of previous generations of PolyhHb. Taken together, these results demonstrate that PolyhHb has the ability to be seamlessly manufactured at the pilot scale to enable future large animal studies

### Introduction

Allogenic blood transfusion is the most definitive treatment for blood loss. Unfortunately, the American Red Cross is experiencing one of the worst blood shortages in over a decade because of the COVID-19 pandemic. This has caused deferment of life-saving procedures such as organ transplants for some patients. Unfortunately, blood shortages are not limited to pandemics, and can occur seasonally and during other crises such as natural disasters or wars. In these situations, blood availability may not meet the demand for blood, or blood may not be available near to the trauma site. To address these challenges, there needs to be alternatives to blood that can keep an individual alive to bridge the gap between blood loss and definitive blood transfusion.

Given the scarcity of blood, the current standard of care administered by emergency personnel involves transfusion of crystalloid or colloid solutions to maintain mean arterial pressure and blood volume. These emergency transfusions are only meant to alleviate the immediate effects of hypovolemic shock and do not provide oxygen (O₂) to tissues, unlike blood. Therefore, hemoglobin (Hb)-based O₂ carriers (HBOCs) are actively being developed as an alternative to crystalloid or colloid solutions as a red blood cell (RBC) substitute to replace lost blood volume. HBOCs are able to resuscitate hypoxic tissues, which is facilitated by Hb's innate ability to bind and release oxygen (O₂). A solution of acellular (cell-free) Hb cannot be used in transfusion medicine as a suitable HBOC, since the protein is small enough (64 kDa) to extravasate through the pores lining the blood vessel wall into the tissue space. As a result, cell-free Hb scavenges nitric oxide (NO), eliciting vasoconstriction and systemic hypertension. In addition, Hb deposition into the tissue space leads to oxidative tissue injury.

To mitigate the cytotoxic effects of cell-free Hb, HBOCs require modification of Hb via processes such as chemical crosslinking or particle encapsulation to generate a molecule/particle large enough to prevent tissue extravasation, while still maintaining Hb's native O₂ transport properties. Polymerized Hb (PolyHb) is the most well-studied HBOC and typically uses glutaraldehyde as a non-specific Hb crosslinker. PolyHeme® (Northfield Laboratories Inc., Evanston, IL), Hemopure® (HbO₂ Therapeutics, Souderton, PA), and Hemolink® (Hemosol, Toronto, ON, Canada) are all commercial PolyHbs that failed phase III clinical trials. These HBOCs are mostly composed of low molecular weight (LMW) Hb species (<500 kDa) that pose similar side-effects to cell-free Hb, thus resulting in their failure in clinical trials. Thus, despite their clinical potential, there are still no FDA approved HBOCs for use in transfusion medicine.

To minimize the adverse side-effects observed in PolyHb clinical trials derived by the presence of LMW polymer species, LMW polymer species can be removed from the PolyHb product via tangential flow filtration (TFF). Building on the success of the bench-top scale polymerized human Hb (PolyhHb) synthesis protocol that can produce 15 g of PolyhHb in one batch, this example focuses on scaling up PolyhHb production to the 200-300 g scale that brackets PolyhHb between 500 kDa and 0.2 μm, therefore removing the majority of LMW polymeric species and cell-free Hb that could elicit vasoconstriction, systemic hypertension, and oxidative tissue injury; as well as, any high molecular weight (HMW) species over 0.2 μm in size that could signal the reticuloendothelial system (RES). Therefore, PolyhHb scaleup is essential to enable safety and efficacy studies in large animals before subsequent evaluation in humans

In this example, PolyhHb scaleup followed a protocol mirroring the PolyhHb bench-top synthesis parameters. The scale of production was increased from the 1.5 L bench-top scale reactor system to a 30 L pilot-scale reactor system. The use of TFF modules enabled scalable purification of PolyhHb. Therefore, this example describes the synthesis and characterization of pilot scale PolyhHb produced in both the low and moderate O₂ affinity state and compares the pilot scale PolyhHb product to previously published bench-top scale PolyhHb product.

### Materials and Methods

Materials. Sodium dithionite (Na₂S₂O₄), glutaraldehyde (C₅H₈O₂) (70 wt %), sodium cyanoborohydride (NaCNBH₃), sodium lactate (NaC₃HO₃), N-acetyl-L-cysteine (NALC, C₅H₉NO₃S) and calcium chloride dihydrate (CaCl₂)·2H₂O) were purchased from Sigma Aldrich (St. Louis, MO). Sodium chloride (NaCl), potassium chloride (KCl), sodium phosphate monobasic (NaH₂PO₄), sodium phosphate dibasic (Na₂HPO₄), sodium hydroxide (NaOH), and 0.2 μm Titan3 sterile filters were purchased from Fisher Scientific (Pittsburgh, PA). Hollow fiber tangential flow filter (TFF) modules N02-P500-05-N (polysulfone (PS), 500 kDa pore size), N02-S20U-05-N (polyethersulfone (PES), 0.2 μM pore size), and N02-E65U-07-N (polyethersulfone (PES), 0.65 μM pore size) were purchased from Repligen (Rancho Dominguez, CA). The Liqui-Cel™ EXF Series G420 Membrane Contactor was purchased from 3M (St. Paul, MN). A minicentrifuge (50-090-100, with a working speed of 6,000 rpm and maximum speed of 6,600 rpm) was obtained from Fisher Scientific. Expired human RBC units were acquired from Transfusion Services at the Wexner Medical Center (Columbus, OH), Canadian Blood Services (Ottawa, Canada), and Zen-Bio Inc. (Durham, NC).

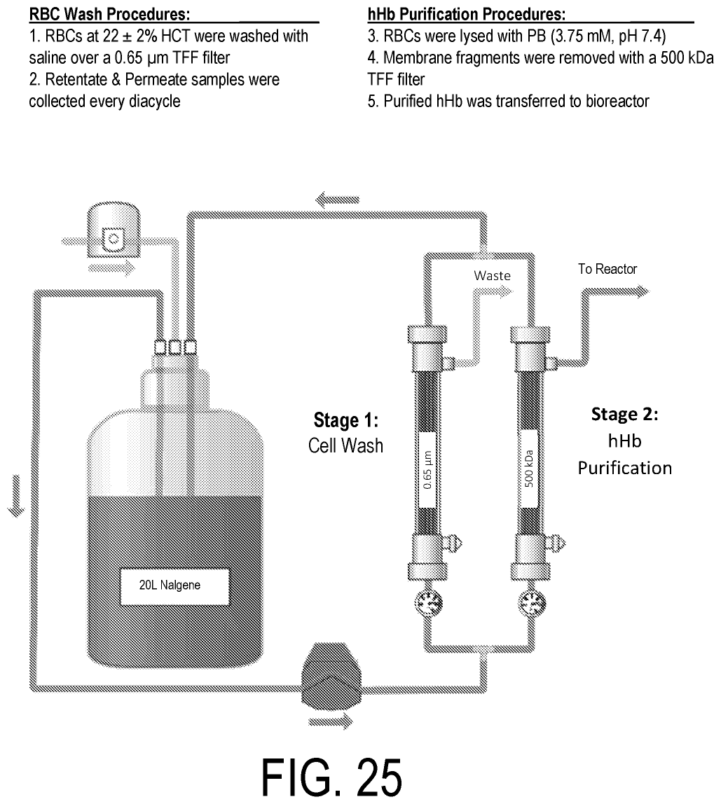

RBC Washing Process. 180 L of 0.9 wt % saline was prepared the night prior to the start of the RBC washing process. To prepare the saline, a vessel was first filled with 176 L of deionized (DI) water followed by the addition of 4 L of 40.5 wt % saline. The vessel was then stirred for 30 minutes and stored at 4° C. For each batch of pooled RBCs, a total of 18 RBC units were used in the washing process. Initially, the system vessel was primed with 0.9 wt % saline before addition of the RBC units. All RBC units were gently inverted and massaged to properly mix blood bag contents and then transferred into a 20 L Nalgene container in a biosafety cabinet and an initial pooled sample of RBCs was taken for analysis. The vessel was then transferred into a chromatography refrigerator, where it was maintained at 4° C. for the entirety of the RBC washing process. The vessel containing pooled RBCs was diluted with 0.9 wt % saline to obtain a 22±2% hematocrit (HCT) to reduce RBC solution viscosity and standardize the HCT between replicates. Once the HCT was confirmed, the permeate line in the TFF RBC washing system (FIG. 25) was opened to initiate the diafiltration process. The diluted RBC solution underwent six system volume exchanges (diacycles) over a 0.65 μm mPES TFF cartridge with 0.9 wt % saline as the wash solution as shown in FIG. 25. Retentate and permeate samples were taken after each diacycle, using a sample port in the retentate line to obtain the retentate sample, while permeate samples were collected from the permeate line entering a waste vessel.

HCT Quantification. RBC solution HCT was determined by loading 65 μL of the retentate sample into a 75 mm mylar wrapped capillary tube (Drummond, Broomall, PA) followed by centrifugation in a Sorvall Legend micro 17 microcentrifuge (Fisher Scientific, Waltham, MA) at 17 g for 5 minutes. The HCT was determined using a standardized HCT calibration curve.

RBC Cell Concentration Quantification. The cell concentration for retentate samples taken during the RBC washing process was measured using a Multisizer 4e Coulter Counter (Beckman Life Sciences, Indianapolis, IN). RBC samples were diluted 100-followed by the addition of 100 μL of the diluted sample into 20 mL of filtered Isoton solution (Beckman Life Sciences) for cell concentration analysis in the Coulter Counter.

Hb Quantification. The cell-free Hb concentration in both permeate and retentate samples from the RBC washing process was measured using UV-visible absorbance spectrometry on a diode array spectrophotometer HP 8452A (Olis, Bogart, GA). Retentate samples were first centrifuged on a minicentrifuge (Fisher Scientific, Waltham, MA) at 6,000 RPM for 2 minutes, followed by removal of the supernatant for spectrometry measurements. The supernatant Hb concentration was analyzed by the cyanomethemoglobin method.

hHb Purification. Post wash, RBCs were lysed with phosphate buffer (PB) (3.75 mM, pH 7.4) for 1 hour at 4° C. with constant stirring. Lysed RBC membrane fragments and large aggregates were removed using a 500 kDa TFF module (FIG. 25). Purified hHb and other RBC proteins<500 kDa were transferred into the stirred reactor shown in FIG. 26. Once 480 g of hHb was loaded into the reactor vessel, a 2 L NaCl charge was added to convert the PB into phosphate buffered saline (PBS). The hHb in the reactor was recirculated through a gas contactor loop in addition to having a nitrogen (N₂) head space in the reactor to deoxygenate the protein. The hHb solution was left to deoxygenate overnight at 14° C. using a cooling coil system to limit methemoglobin (metHb) formation.

hHb Polymerization. The next day, the hHb solution in the reactor was brought up to physiological temperature (37° C.) using a thermal jacket wrapped around the reactor. The pO₂ of the system was checked periodically using a RAPIDLab 248 blood gas analyzer (Siemens, Munich, Germany) with the goal of achieving a pO₂ value of 0.0±2.0 mmHg before hHb polymerization in order to achieve 100% tense quaternary state (T-state) PolyhHb. To ensure complete hHb deoxygenation, sodium dithionite was added in 1 g charges until a pO₂ of 0 mm Hg was achieved.

Once the hHb in the reactor was fully deoxygenated, the gas contactor was temporarily bypassed to begin hHb polymerization as shown in FIG. 26. Polymerization was performed with approximately a 30:1 molar ratio of glutaraldehyde (GA) to hHb. The volume of GA was diluted in 3 L of PBS (pH 7.4) and bubbled with N₂. The solution was then pumped into the reactor over three hours followed by an additional hour of reaction time. A N₂ head space was maintained in the reactor and the GA solution vessel. Following the hHb polymerization reaction, the heating jacket was removed, and the cooling coils were turned on. The system was quenched with a 7:1 molar ratio of NaCNBH₃ to GA, diluted in 3 L of PBS (pH 7.4). The quenching solution was added to the reactor over a period of 10 minutes. The system was monitored for at least 30 minutes before being held at 14° C. overnight. Throughout the hHb polymerization process, the impeller speed was maintained at approximately 130 rpm and turned off during the overnight hold time.

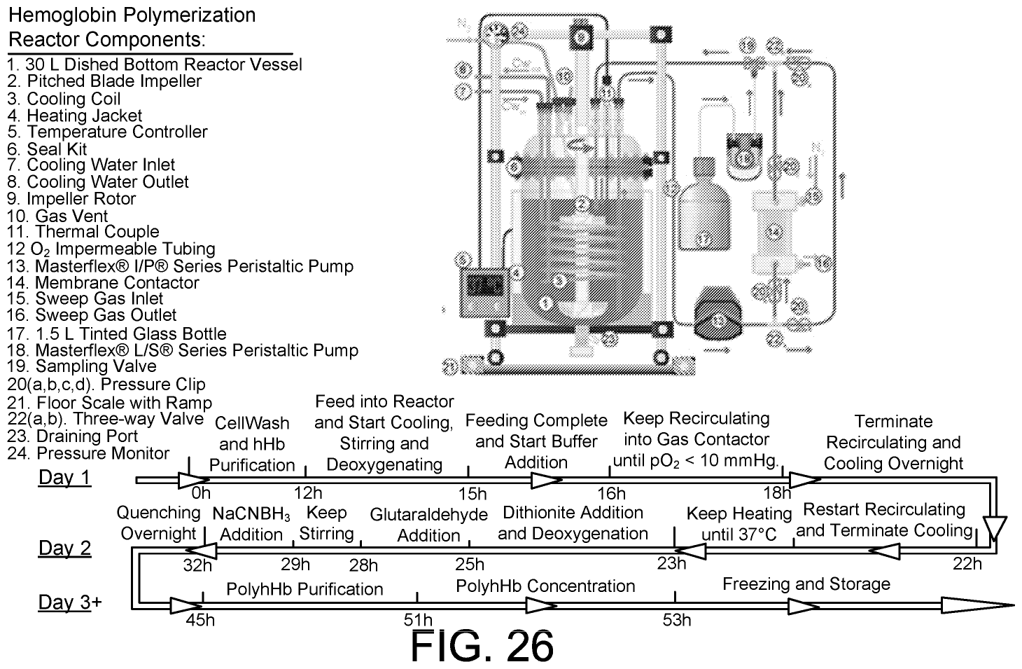

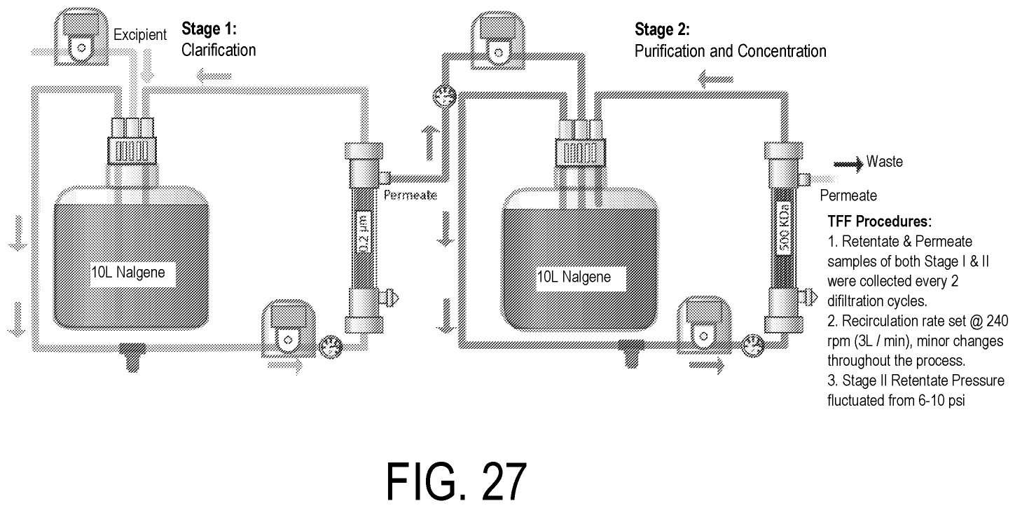

PolyhHb Separation and Purification. Following the hHb polymerization process, the PolyhHb solution was pumped from the reactor to a stage 1 PolyhHb purification vessel into a chromatography refrigerator as shown in FIG. 27. The stage 1 recirculation loop passed the PolyhHb solution through a 0.2 μm PES TFF cartridge into a stage 2 PolyhHb purification vessel as shown in FIG. 27, with undesired particulates or high MW polymerized material being retained in stage 1 (>0.2 μm). The stage 2 recirculation loop facilitated excipient exchange of the PolyhHb solution over a 500 kDa PS TFF cartridge with a modified Ringer's lactate solution. This was performed to both remove LMW Hb polymeric species (<500 kDa), unmodified hHb, and free reactants from the final product as well as to exchange the PolyhHb from the PBS buffer to a more physiologically relevant crystalloid (modified Ringer's lactate). At least twelve full diacycles were performed to ensure the final PolyhHb solution contained <5% LMW Hb species (<500 kDa). Once the excipient exchange was completed, the final PolyhHb product was concentrated to over 10 g/dL. The final protein concentration and metHb level were measured using the cyanomethemoglobin method (31). The final PolyhHb product was stored at −80° C. until use.

Oxygen Equilibrium Curve. A Hemox Analyzer (TCS Scientific Corp., New Hope, PA) was used to measure the oxygen equilibrium curve (OEC) of hHb and PolyhHb. The OEC was fit to the Hill equation to regress the cooperativity coefficient (n_{H}) and partial pressure of O₂ at which 50% of the hHb or PolyhHb was saturated with O₂ (P₅₀).

Polymer MW and Size. The MW distribution of hHb and PolyhHb was measured using size exclusion chromatography (SEC) on a high-pressure liquid chromatography (HPLC) system. The separation was performed using an Ultimate 3000 system with an SEC-1000 column (ThermoFisher Scientific, Waltham, MA). The absorbance was measured at 412 nm to monitor the Soret peak characteristic of hHb. The hydrodynamic diameter of hHb and PolyhHb was measured using a Zetasizer Nano Dynamic Light Scattering (DLS) Spectrometer (Malvern Instruments, Worcestershire, UK).

Auto-Oxidation Kinetics. UV-visible absorbance spectrometry was used to measure the auto-oxidation kinetics of hHb and PolyhHb. The hHb/PolyhHb solution was diluted in PB to 12.5 g/L to simulate the average concentration of product in the systemic circulation following transfusion. The solution was monitored for 24 hours, while maintaining a physiological temperature (37° C.) and pH (7.40). The absorbance at 630 nm was monitored over a 24-hour period. Analyzing the kinetics for hHb and PolyhHb, the fraction of heme in the ferrous state as a function of time was analyzed assuming first-order rate kinetics to regress the auto-oxidation rate constant (kₒₓ) in h⁻¹.

Stopped-Flow Kinetics. A SX-20 micro-volume stopped-flow apparatus (Applied Photophysics, Leatherhead, UK) was used to measure the O₂ offloading kinetics and the haptoglobin (Hp) binding kinetics of hHb/PolyhHb. For the O₂ offloading kinetics, the absorbance at 437.5 nm was measured when a 12.5 μM (heme-basis) solution of oxygenated hHb/PolyhHb in PBS was rapidly mixed with a 1.5 M solution of sodium dithionite in degassed PBS. The average of the kinetic traces was fit to an exponential function using the Applied Photophysics software to regress the first-order Oz dissociation rate constant (k_{O2,off}) in s⁻¹.

Hp binding kinetics was monitored by measuring the fluorescence change at 285 nm when Hp binds to hHb/PolyhHb. A 5 μM, 2.5 μM, 1.25 μM, and 0.625 μM solution of hHb/PolyhHb sample in PBS was rapidly mixed with a 0.25 μM solution of Hp. The average of the kinetic traces was fit to an exponential function and a pseudo-first order rate constant was regressed at each concentration. The pseudo-first order rate constants were plotted against hHb/PolyhHb concentration and fit to a linear function to regress the second-order Hp binding rate constant (k_{Hp-Hb}) in μM⁻¹s⁻¹.

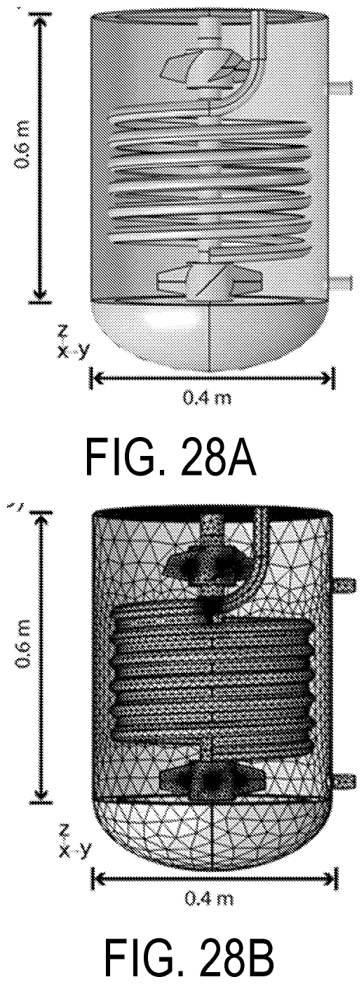

Computational Methods. The fluid dynamics and mixing performance in the PolyhHb reactor vessel was modeled as a continuous stirred tank reactor (CSTR) and evaluated via Comsol Multiphysics (Version 5.3a, Comsol, Inc., Burlington, MA). In this example, the Rotating Machinery module was used to solve for the turbulent flow properties in the reactor. The simulated results were presented at steady state. Constant circulation of PolyhHb solution was maintained in between the inlet and outlet as shown in FIGS. 28A-28B via a peristatic pump. The CSTR contained two 4-bladed pitched impellers, and a cooling coil. Fluid flow was evaluated using a stationary MUMPS solver with a relative tolerance of 1.0E-6 and automatic linearity. All simulations were conducted in 3D using a circular uniform distribution.

Statistical Analysis. All data is presented as the mean±standard deviation. Statistical analysis for data collected in the study was performed on RStudio using t-tests. For all tests, p<0.05 was considered statistically significant.

### Results and Discussion

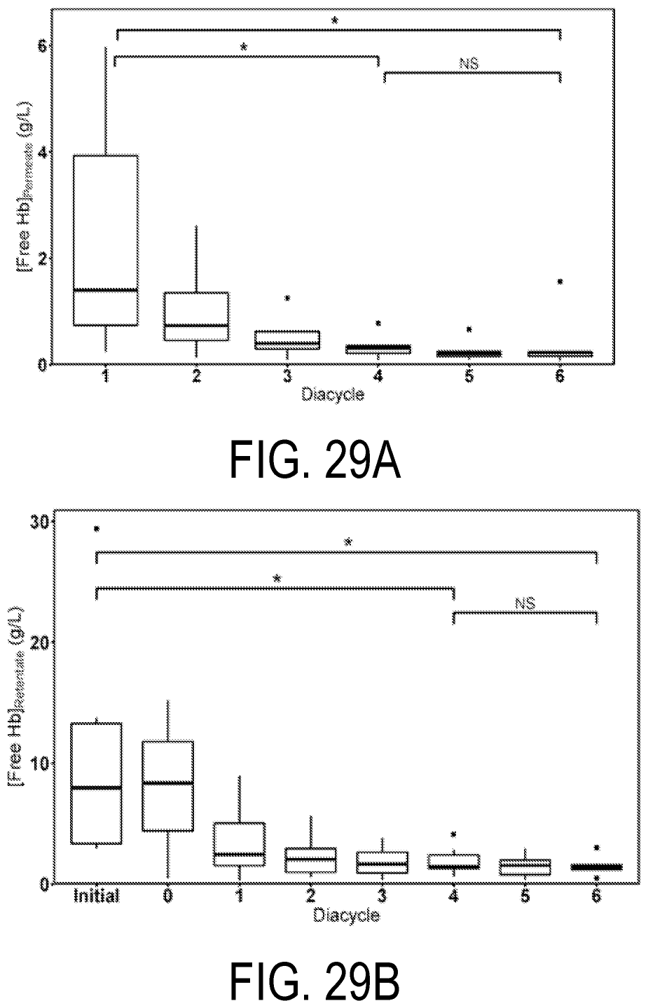

RBC Washing Process. The RBC washing process successfully removed the majority of cell-free Hb from the expired pooled RBC units, while maintaining the more mechanically resilient RBCs for use in the subsequent hHb purification step of the pilot scale hHb polymerization process. FIG. 29A shows that after 4 diacycles, the concentration of cell-free Hb in the permeate dropped below 0.5 g/L and remained constant for the remaining diacycles, verifying that the RBCs were thoroughly washed, while the more mechanically susceptible RBCs lysed and were removed from the system. Similarly, FIG. 29B shows that after 4 diacycles, the concentration of cell-free Hb in the retentate remains below 2 g/L, which indicated that as much cell-free Hb was removed as possible from the system.

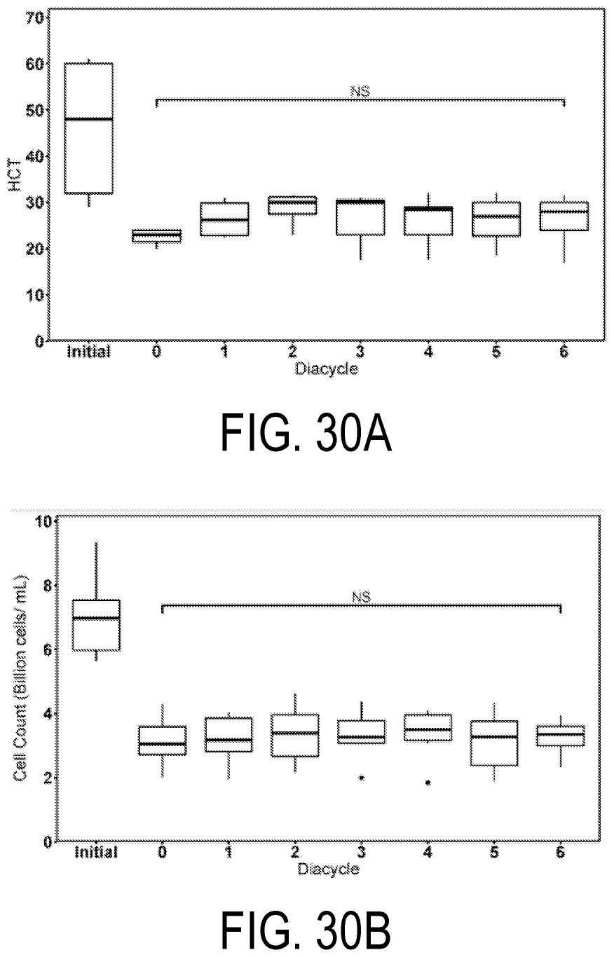

The HCT is defined as the volume fraction of packed RBCs present in the RBC solution and is directly related to the concentration of RBCs in a well-mixed solution. In FIG. 30A, the HCT was measured for the retentate after each washing diacycle to determine the volume fraction of intact RBCs present in the system volume during the RBC washing process. It is possible to wash RBCs with negligible hemolysis at physiological HCTs using a TFF system driven by a centrifugal pump. Conversely, peristaltic pumps are known to lyse cells traversing through the pump head due to tubing compression and subsequent pressure applied to the cells, which would decrease the HCT during the RBC washing process. However, in this example, the HCT remained constant (p=0.993) as shown in FIG. 30A after the initial HCT adjustment from the pooled packed RBC units to a HCT of 20-24%, which indicated that with appropriate RBC dilution and application of a low pump speed, it is possible to wash RBCs with negligible loss due to shear-induced hemolysis.

The concentration of intact RBCs was then used to determine whether washing RBCs using TFF and subjecting RBCs to shear forces during the washing process is potentially detrimental to cell integrity and causes more unintentional hemolysis. No significant drop in RBC concentration was observed (p=0.999) between the first and sixth diacycles as shown in FIG. 30B, which further confirms the HCT measurements which indicated that there is negligible hemolysis during the RBC washing process at the selected pump speed.

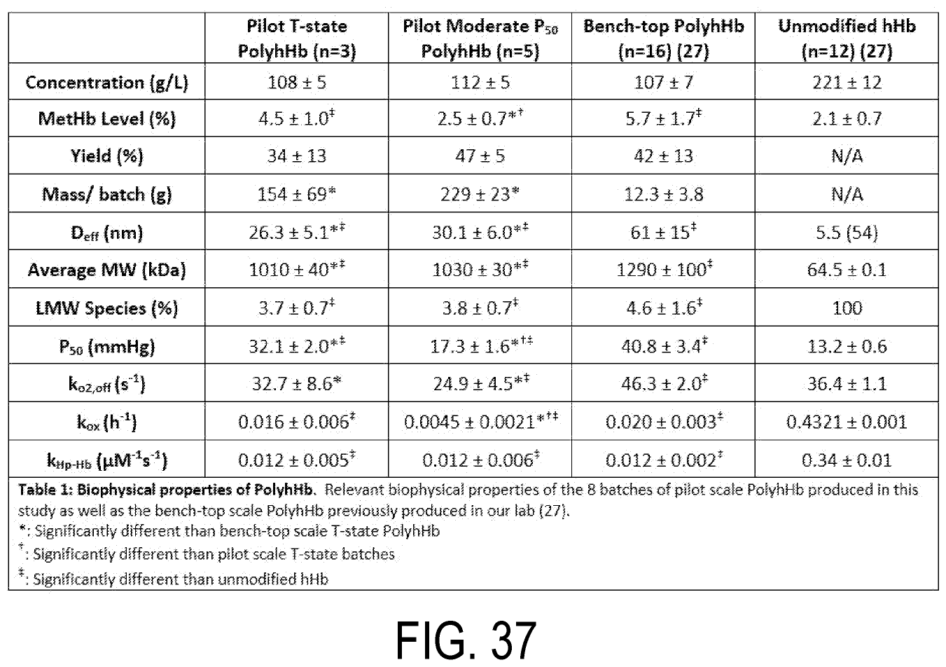

PolyhHb Quantification. FIG. 37 summarizes the various biophysical parameters measured in this study as well as values for 30:1 T-state PolyhHb batches previously produced in our lab at the bench-top scale. The eight pilot scale batches in this study were further subdivided into three fully T-state batches and five moderate P₅₀ batches (where the quaternary state of the Hb in the PolyhHb molecules is between that of T-state and relaxed quaternary state (R-state) Hb).

All pilot scale PolyhHb batches produced in this example were concentrated to at least 100 g/L. This is in line with the protocol for PolyhHb production used in our lab for bench-top scale PolyhHb production. Additionally, the average yield for pilot scale PolyhHb batches was 42%, which is identical to the yield of previous bench-top scale 30:1 T-state PolyhHb batches produced in our lab. In summary, the scaled-up PolyhHb production process does not result in a loss in yield compared to the bench-top scale process. On average, the pilot scale process produced 201 g of PolyhHb—16.3× more compared to the benchtop scale—with moderate P₅₀ batches producing 18.6× as more compared to the bench-top scale. This exponential increase in PolyhHb production in a single batch helps to both validate the success of the scaled up process, and gives promise to the use of the next-generation PolyhHb in future large animal studies.

The most significant difference in protein quantification between pilot scale and bench-top scale PolyhHb process is the percentage of metHb (i.e., metHb level) in the PolyhHb product. The PolyhHb produced in this example included on average 3.2% metHb, which is significantly lower compared to the 30:1 T-state PolyhHb produced previously at the bench-top scale (p=0.0014). Upon analysis of the two subgroups, this trend appears to be due to the lower metHb level of the moderate P₅₀ pilot scale batches. The five moderate P₅₀ pilot scale batches have a significantly lower metHb level than the three fully T-state batches produced at the pilot scale (p=0.015). This is likely due to two separate factors. First, the pilot-scale PolyhHb synthesis protocol had the purified hHb added directly to the reactor after RBC lysis and filtration through a 500 kDa TFF module. In the bench-top scale synthesis of PolyhHb, the hHb was first filtered through a 500 kDa TFF module and then further concentrated over a 50 kDa TFF module for subsequent storage at −80° C. before use. The final concentration step on the 50 kDa TFF module removes excess buffer as well as any proteins<50 kDa, including superoxide dismutase (SOD) which is critical in preventing Hb oxidation. Co-polymerizing SOD with PolyhHb can limit the oxidation of PolyhHb, and a similar mechanism is likely at play here to reduce the metHb level of all pilot-scale batches compared to bench-top scale PolyhHb. Second, the low O₂ affinity (i.e., high P₅₀) of T-state PolyhHb leads to a higher rate of metHb formation compared to species with lower P₅₀s. This is discussed more in later sections, but for now it would help explain the difference in metHb level between moderate P₅₀ and fully T-state PolyhHb pilot scale batches.

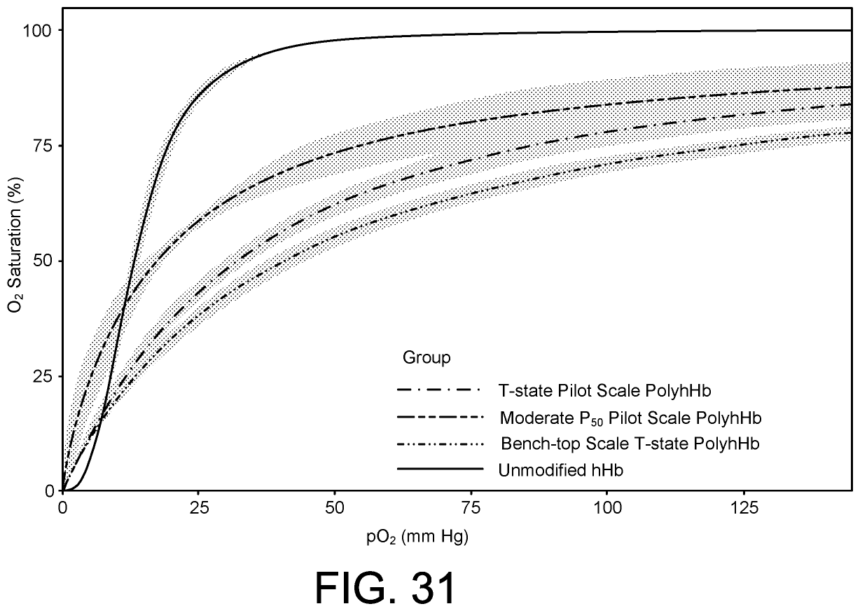

O₂ Equilibria. The PolyhHb produced in this study fell into two distinct groups: T-state PolyhHb and a moderate O₂ affinity PolyhHb, as shown in FIG. 31. Traditionally, PolyhHb has been synthesized in either a low oxygen affinity T-state (P₅₀>30 mm Hg) or a high oxygen affinity R-state (P₅₀<2 mm Hg). A moderate P50 PolyhHbs (P₅₀: 10-20 mm Hg) can be prepared by mixing R-state and T-state fractions together to create a solution with a P₅₀ closer to that of unmodified hHb (12.4 mm Hg). In this example, three pilot scale batches were fully deoxygenated before glutaraldehyde addition and throughout the polymerization process resulting in the final PolyhHb product being fully in the T-state. However, five of the batches were not kept under fully anoxic conditions during the polymerization process, with the O₂ tension of the reactor getting as high as 5 mm Hg before quenching the polymerization reaction. The marginal amount of O₂ in the system—only about 3% of the O₂ tension of air—alters the quaternary state of the hHb solution to exist in both a partially oxygenated and partially deoxygenated state. This mixture of quaternary states is locked into place during polymerization, creating the moderate P₅₀ PolyhHb species observed in this example.

T-state PolyhHb is traditionally used for hemorrhagic shock resuscitation and other therapeutic applications. The low O₂ affinity of T-state PolyhHb results in the ability of O₂ to be readily offloaded to surrounding tissue to reduce tissue hypoxia. More recent studies have shown that the indiscriminate offloading of O₂ by T-state PolyhHb may not be as beneficial as originally thought. For instance, there is an increase in reactive oxygen species (ROS) generation under hyperoxic conditions. This triggers a cascade of inflammatory response pathways, leading to increased cellular damage. Additionally, the principle of autoregulation—whereby in the presence of an O₂-rich environment, cells will automatically reduce O₂ uptake—has been shown to limit the efficacy of Oz carriers with P₅₀s significantly higher than RBCs (26 mm Hg). It is for these reasons that moderate P₅₀ PolyhHbs show promise as viable RBC substitutes with reduced potential for ROS generation and autoregulatory response.

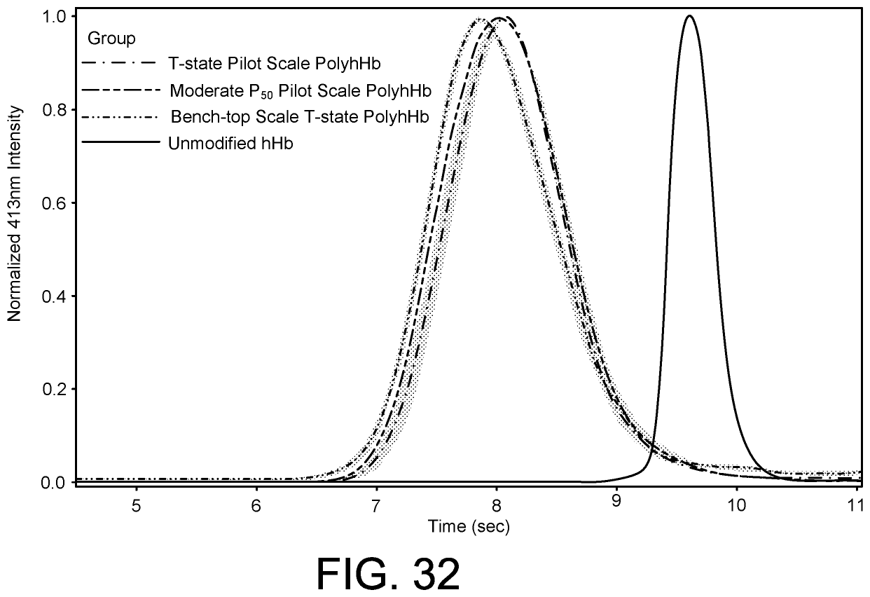

Size Distribution. The size of the PolyhHb synthesized in this example was measured using both DLS and SEC-HPLC and the SEC-HPLC results are shown in FIG. 32. DLS revealed an average effective diameter (Deff) of 29.6 nm (FIG. 37). This is significantly less than PolyhHb of the same crosslink density produced at the bench-top scale (p<0.001). Similarly, SEC-HPLC revealed that the PolyhHb produced at the pilot-scale is smaller than bench-top scale 30:1 T-state PolyhHb. The pilot scale PolyhHb produced in this study had an average MW of 1020 kDa which is significantly smaller than the 1290 kDa average MW of bench-top scale PolyhHb (p<0.001). The PolyhHb synthesized in this example is still classified as an ultra-high MW (MW≥1000 kDa), high-purity PolyhHb so the size disparity is of no concern.

The size difference between pilot scale and bench-top scale batches is likely due to mixing differences between the pilot-scale reactor system compared to the bench-top scale reactor system. Bench-top PolyHb syntheses used a stir bar as the agitator for mixing and the reactor has no baffles, while the pilotscale reactor is a baffled CSTR with an impeller, so it inherently generates more uniform mixing. Ostwald ripening is a well-described process whereby particles in solution are more likely to interact with larger particles over smaller ones. In a reactor set-up where mixing is not as uniform as in a CSTR, it is to be expected that Ostwald ripening will occur, resulting in a more bimodal distribution of Hb polymers. This is opposed to a perfectly mixed system, where there should be a more even distribution of polymer sizes, which would explain the smaller MW of the PolyhHb produced at the pilot-scale compared to bench-top scale. The optimal mixing of the pilot scale reactor is confirmed in the computational fluid modeling described in a later section.

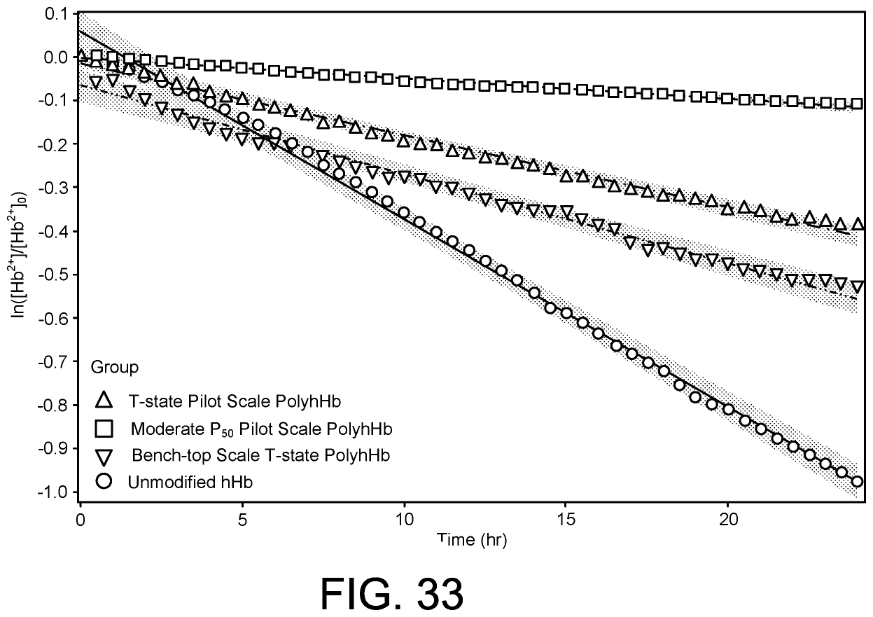

Auto-Oxidation. Similar to the discussion regarding PolyhHb oxygen affinity, the auto-oxidation rate constant of pilot scale PolyhHbs produced in this study fell into two distinct groups. The auto-oxidation rate constant and oxygen affinity appear to be mildly correlated in FIG. 33. Every pilot scale batch with a moderate P₅₀ resulted in a lower auto-oxidation rate constant, and most of the T-state PolyhHb batches fell into the higher autooxidation rate constant group. Examining the subgroups as a whole, the moderate P₅₀ pilot scale PolyhHbs exhibited statistically significantly lower auto-oxidation rate constants than fully T-state pilot scale PolyhHbs (p=0.0065). This phenomenon was observed previously, with R-state PolyhHb exhibiting a lower auto-oxidation rate constant compared to T-state PolyhHb.

The kₒₓ for both pilot scale groups in this study were lower compared to other HBOCs in the literature. Unmodified hHb has been shown to have a kₒₓ of 0.043 h⁻¹, which is 2× higher than the pilot scale T-state PolyhHb group and ˜9× higher than the moderate P₅₀ pilot scale PolyhHb group. Furthermore, the commercial PolyhHb PolyHeme® (Northfield Labs, Evanston, IL) was shown to have a kₒₓ of 0.26 h⁻¹, which is 10-fold and 45-fold higher than the pilot scale PolyhHbs synthesized in this example. The low concentration of LMW Hb polymers (<500 kDa) present in the pilot scale materials is thought to be the reason for the lower auto-oxidation rate constant compared to prior generations of HBOCs. A positive correlation between the concentration exists between low MW species in the PolyhHb solution and the auto-oxidation rate constant. All PolyhHbs synthesized in this example had less than one tenth of the LMW species (<500 kDa) that were present in previous commercial HBOCs, so it makes sense for the auto-oxidation rate constant to also be an order of magnitude lower as well.

The lower auto-oxidation rate constant is important for two major reasons. The first is that a lower rate of auto-oxidation will correspond to a greater amount of ferrous PolyhHb being capable of carrying and offloading O₂ while in circulation. When ferrous PolyhHb oxidizes, it converts into the ferric state (i.e., metHb), and is no longer able to bind and release O₂. For HBOCs with high kₒₓ values, the O₂-carrying capacity of the solution drops off rapidly as the ferrous HBOC converts to metHb and therefore loses functionality over a very short time period. In contrast, low kₒₓ HBOCs, such as the pilot scale PolyhHb produced in this example, do not have this problem and remain functional for a much longer period of time, making them significantly more viable as an O₂ therapeutic. Additionally, the Fe³⁺ in metHb is a strong oxidizer, which gives it the ability to produce ROS that are cytotoxic. This further supports the advantages of the low kₒₓ HBOCs produced in this study as these molecules will be less likely to catalyze ROS production and elicit cellular toxicity.

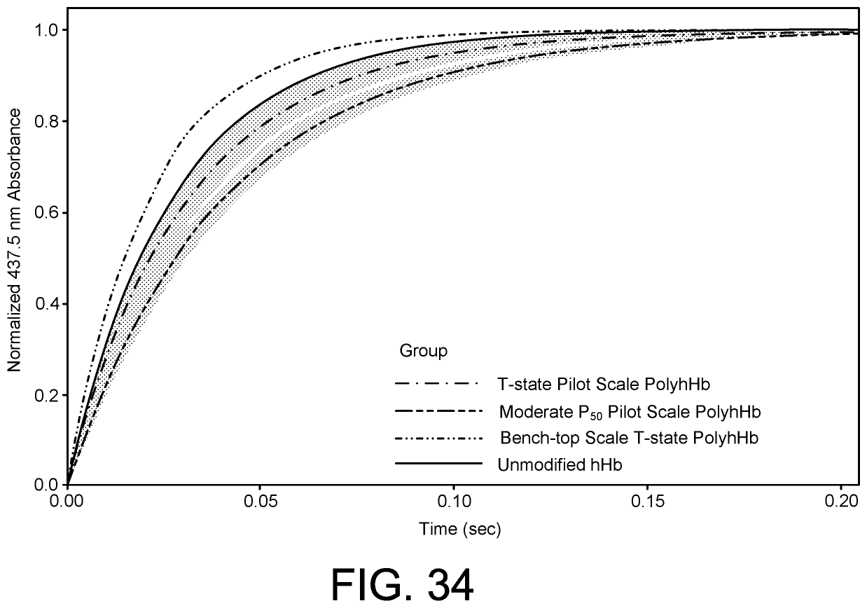

Deoxygenation Kinetics. FIG. 34 shows the kinetics of O₂ offloading by PolyhHb produced at the pilot-scale was lower than Tstate PolyhHb produced at the bench-top scale (p<0.001). Initially this was assumed to be related to the lower average P₅₀ of pilot scale PolyhHb compared to bench-top scale PolyhHb. Previous studies have shown that k_{O2,off} decreases with a decrease in P₅₀ with T-state PolyhHb having a k_{O2,off}>41 s-1 and R-state PolyhHb having a k_{O2,off}<30 s⁻¹. This was not the case in this example though, with 2 of the 3 batches of Tstate PolyhHb having a k_{O2,off} less than that of hHb (41 s⁻¹). The initial hypothesis of O₂ affinity and Oz offloading rate constant being related falls apart even further when considering the moderate P₅₀ pilot scale batches of PolyhHb. Despite all 5 moderate P₅₀ PolyhHb pilot scale batches being closer in O₂ affinity to hHb than R-state PolyhHb, they all have a k_{O2,off} resembling that of R-state PolyhHb (˜23 s⁻¹), not hHb. These results speak well to the potential ability of pilot-scale PolyhHb to carry and offload O₂ while preventing autoregulation; however, the cause for the decoupling of O₂ affinity and Oz offloading rate constant is still yet to be determined.

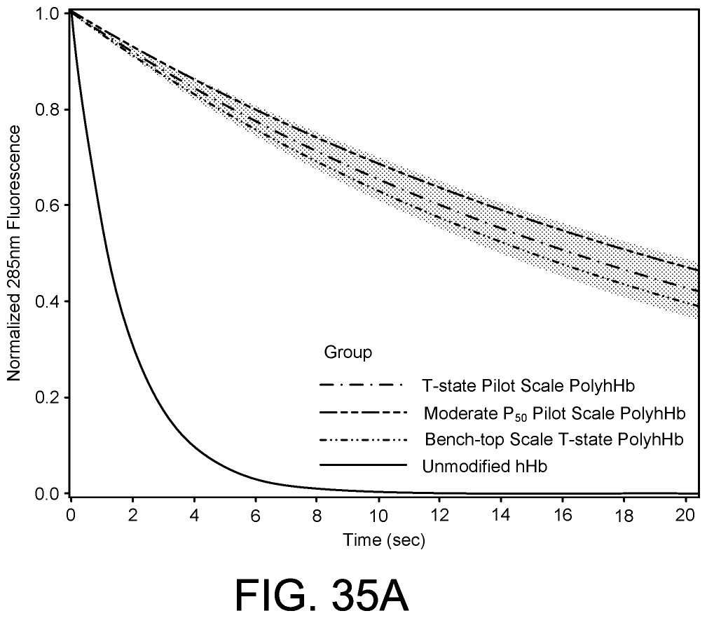

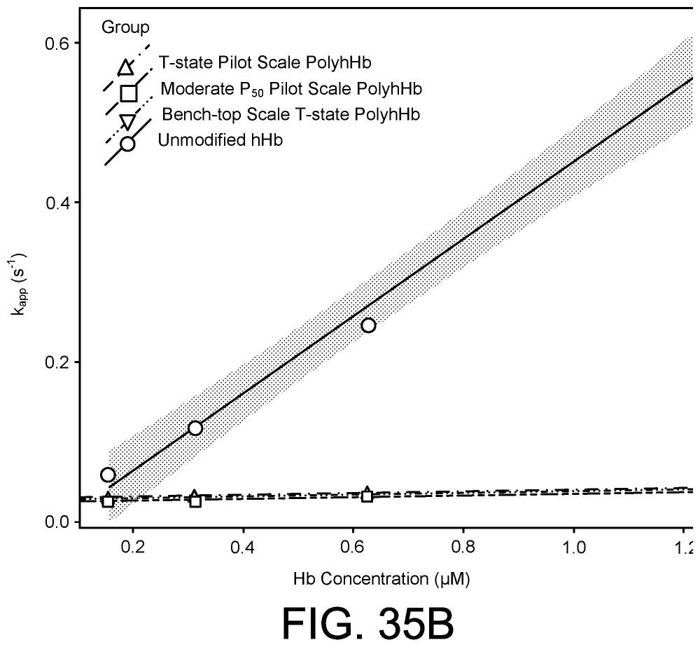

Hp Binding Kinetics. The plot of the pseudo-first order rate constant as a function of Hb concentration for Hp binding (kHp-Hb) to the PolyhHbs produced in this study is shown in FIG. 35B and was identical to bench-top scale PolyhHb. A representative kinetic time course for PolyhHb binding to Hp shows the overlap between PolyhHb groups in FIG. 35A. The reason for the difference in Hp binding rates between hHb and all PolyhHb groups is likely due to the increased measures taken to remove LMW species (<500 kDa) from the final PolyhHb product. Both the pilot-scale PolyhHb produced in this example, and the bench-top scale PolyhHb produced previously were subject to diafiltration over a 500 kDa membrane. Hp can only bind to cell-free Hb and LMW Hb species. Therefore, it is logical that thorough removal of such species through a 500 kDa TFF membrane should drastically reduce the Hp binding rate to PolyhHb. This is supported by the fact that both pilot-scale and bench-top scale PolyhHb have less than 5% LMW species in solution, so they should both have similar, trivially small kHp-Hb values compared to hHb.

Because Hp is the native hHb clearance protein, a high level of Hp-binding would imply the presence of large quantities of the cytotoxic species. The elimination of such species would therefore lead to a decrease in kHp-Hb. This can be taken to mean that kHp-Hb is inversely proportional to the safety of the PolyhHb if it were to be transfused, since a low Hp binding constant would imply less cytotoxic potential. The kHp-Hb of the PolyhHb produced in this example is trivial and 100-fold less than that of unmodified hHb, alluding to its safety as a potential RBC substitute.

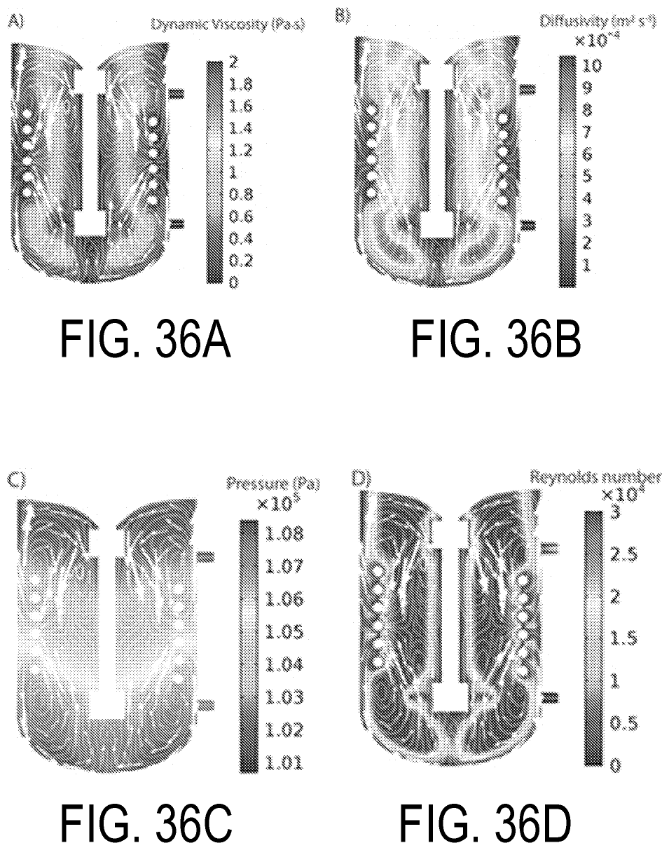

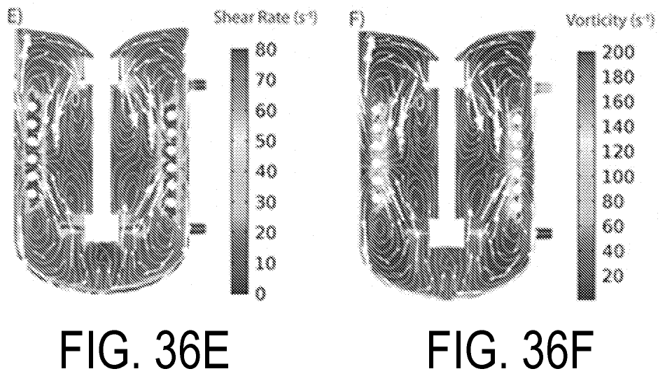

Mixing Modeling. FIG. 36A shows the dynamic viscosity distribution in the pilot scale reactor in the case of 4-bladed pitched impeller at a revolution speed of 130 rpm. The dynamic viscosity in the bulk solution (0.6-1.1 Pa·s) was slightly higher than that in the regions proximate to the interior wall, coil, and impellers (0<0.6 Pa·s) of the reactor. FIG. 36B shows the eddy diffusivity profile in the reactor during the polymerization process. The diffusivity was observed to be maximum (0.001 m² s⁻¹) at the surface of the impeller, which decreased radially towards the vessel wall. It appeared that there was sufficient vertical and horizontal diffusion in the bulk solution (0.0005-0.001 m² s⁻¹). The pressure distribution was shown in FIG. 36C, where a pressure gradient was observed from 101 kPa to 108 kPa. FIG. 36D shows the Reynold's number (Re) distribution in the stirred tank reactor. It is observed that turbulent flow was fully developed in the bulk solution (Re>4000). The solution proximate to the cooling coil and wall exhibited transient flow behavior given that the Re ranged from 2000 to 4000. As expected, the region near the cooling coil and interior wall was not well mixed compared to the bulk of the reactor.

FIGS. 36E and 36F show that the shear rate and vorticity at a rotational speed of 130 rpm were higher near the impeller than in the bulk solution. It was found that the pitched turbine impeller can be used to achieve adequate mixing in the stirred vessel, since negligible vorticity was generated. Vigorous mixing with turbulent flow assisted in preventing vortex formation. Overall, we demonstrate the usefulness of CFD modeling for evaluating the mixing performance in the reactor. Future work should model heat transfer to calculate the temperature profile in the reactor during the polymerization process.

### Conclusions

This example establishes that a high MW next-generation PolyhHb with limited LMW species can be produced at the pilot scale (200-300 g of PolyhHb product). Bench-top and pilot-scale PolyhHb processes exhibited nearly identical protein yields. Additionally, pilot-scale PolyhHb produced in this example was shown to have a lower metHb level and auto-oxidation rate constant compared to bench-top scale PolyhHb. We also demonstrated that PolyhHb could be produced in either the low O₂ affinity T-state or at a moderate P₅₀ making it possible to produce an HBOC that can readily offload O₂ without possible concerns over autoregulation. Optimized mixing parameters most likely lead to the pilot-scale PolyhHb being smaller compared to previous bench-top scale PolyhHbs. Furthermore, the pilot scale PolyhHbs produced in this exhibited a low Hp binding rate constant, further indicating the potential the safety of the material.

### Example 3. Production of Polymerized Human Hemoglobin

In this example, we produced and characterized 12 batches of tense (T) quaternary state polymerized human hemoglobin (PolyhHb) of varying size. The PolyhHbs were then separated into 4 molecular weight (MW) brackets using tangential flow filtration (TFF): 50-300 kDa (PolyB1), 100-500 kDa (PolyB2), 500-750 kDa (PolyB3), and 750 kDa-0.2 μm (PolyB4). Each PolyhHb batch was synthesized using the chemical cross-linker glutaraldehyde (GA) at various cross-link densities to optimize product yield within the designated MW bracket. Specifically, PolyB1 was synthesized at a 10:1 molar ratio of glutaraldehyde to human hemoglobin (hHb) (GA:hHb) and bracketed using a 50 kDa TFF filter and a 300 kDa modified polyethersulfone (mPES) TFF filter. PolyB2 was synthesized at a 25:1 molar ratio of GA:hHb and bracketed using a 100 kDa TFF filter and a 500 kDa mPES TFF filter. Similarly, PolyB3 was produced at a 26.5:1 molar ratio of GA:hHb and bracketed using a 500 kDa polysulfone (PS) TFF filter and a 750 kDa mPES TFF filter. Finally, PolyB4 was synthesized at a 30:1 molar ratio of GA:hHb and the resultant material was bracketed using a 750 kDa filter and a 0.2 μm mPES TFF filter. The bracketed materials were subject to diafiltration with a modified lactated Ringer's solution (pH=7.40) and concentrated to ≥10 g/dL for subsequent use in animal models. The biophysical properties such as the O₂ affinity, O₂ offloading kinetics, haptoglobin (Hp) binding kinetics, total heme concentration, methemoglobin concentration, total protein concentration, effective diameter, and size/MW distribution of each batch were analyzed after preparation.

Our primary goal during the synthesis and production of PolyhHb was to determine the optimal cross-linking density which yielded the target MW bracket with low polydispersity index (PDI).

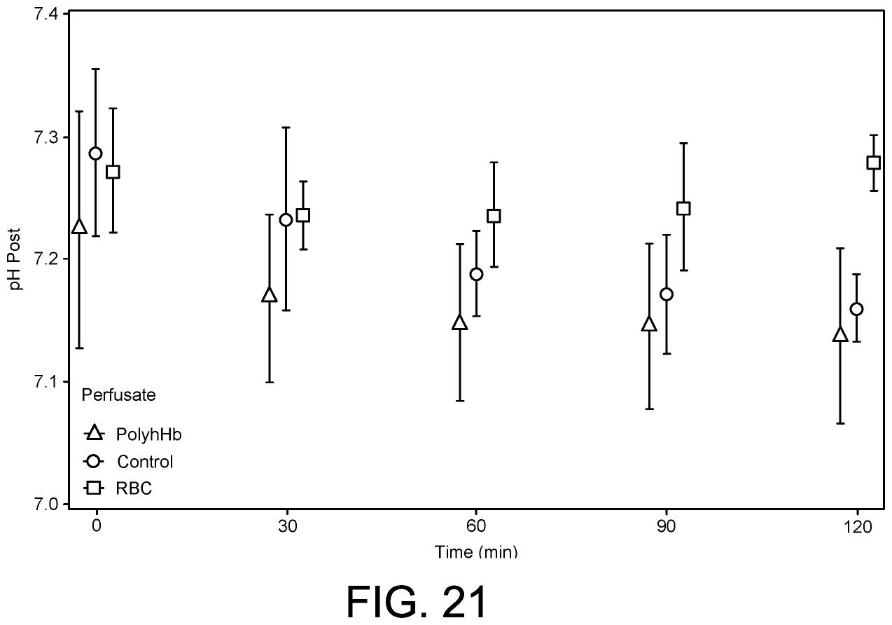

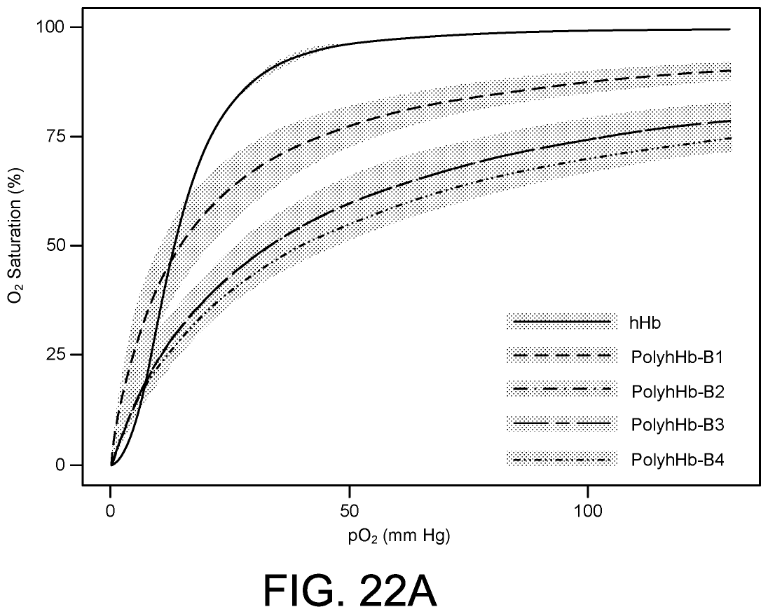

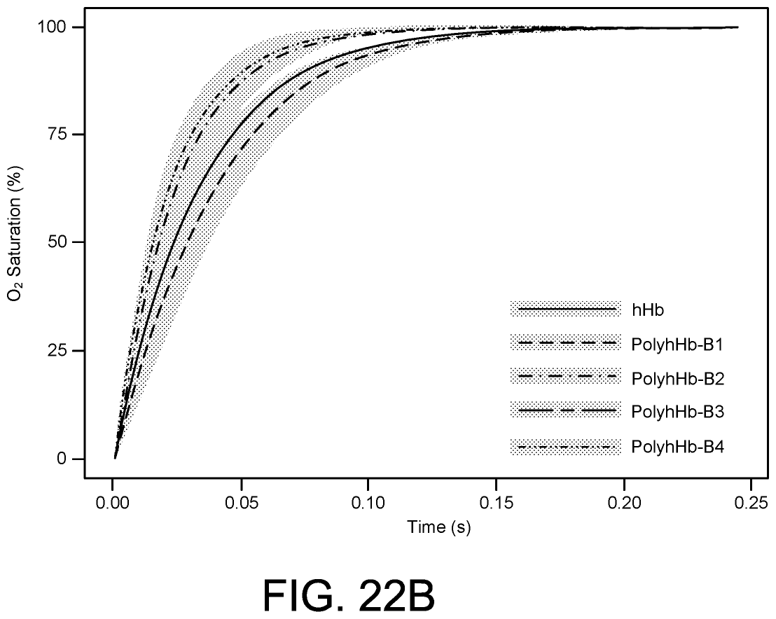

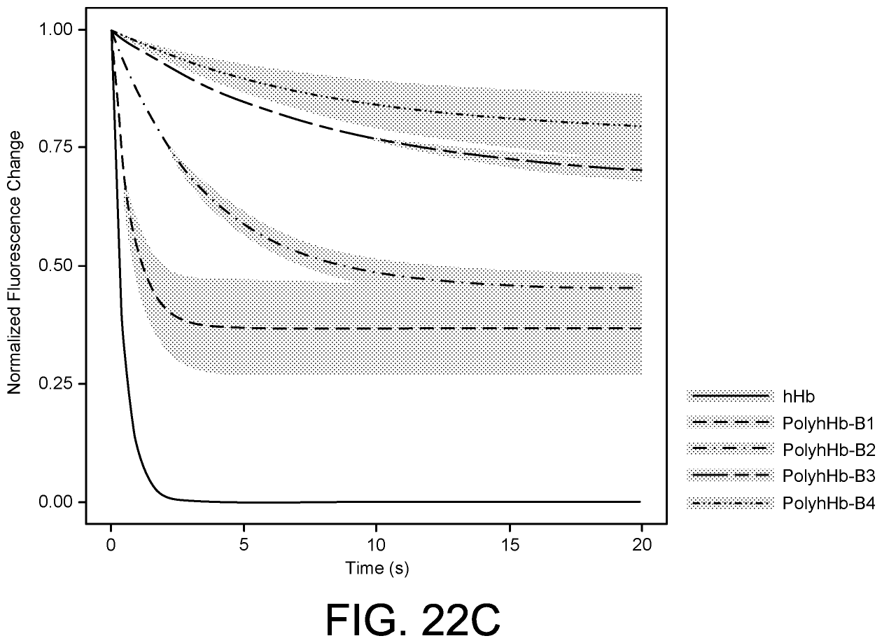

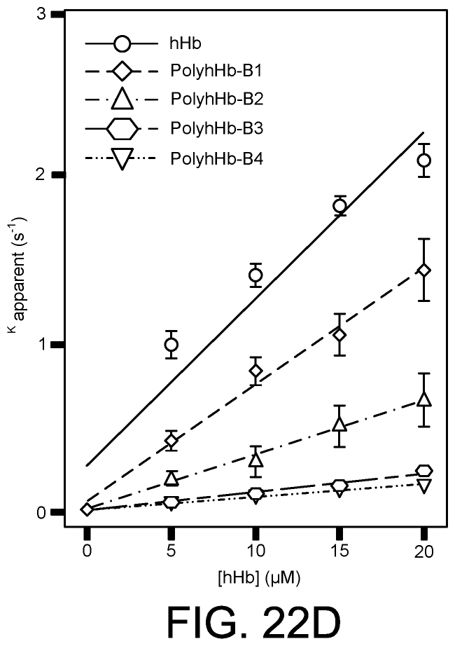

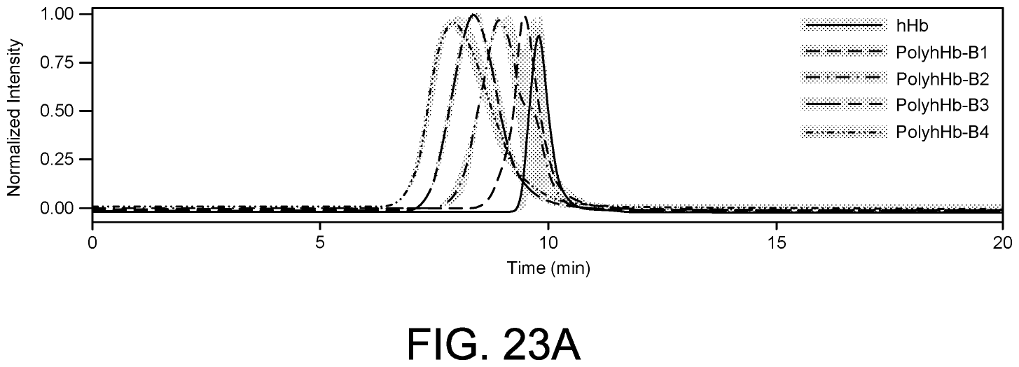

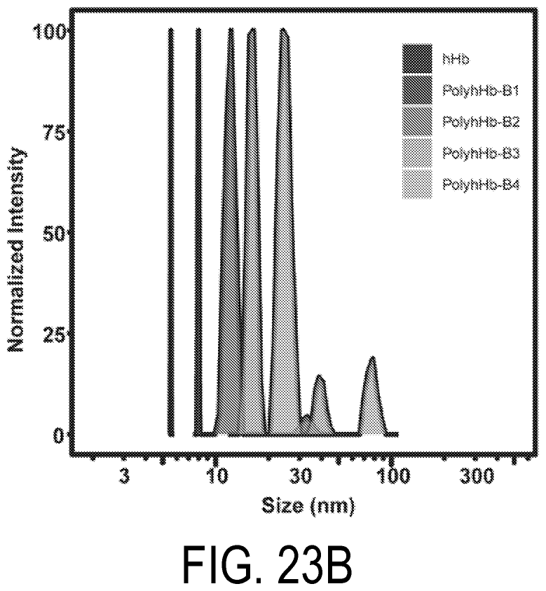

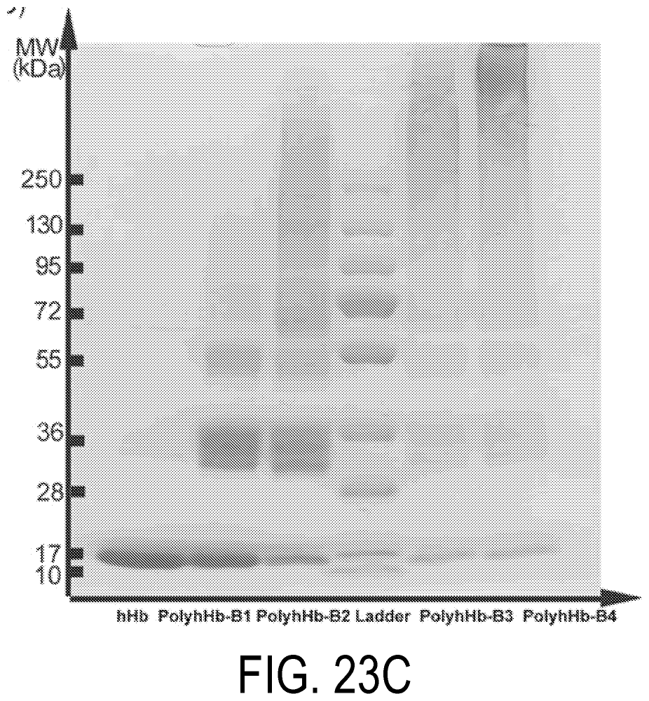

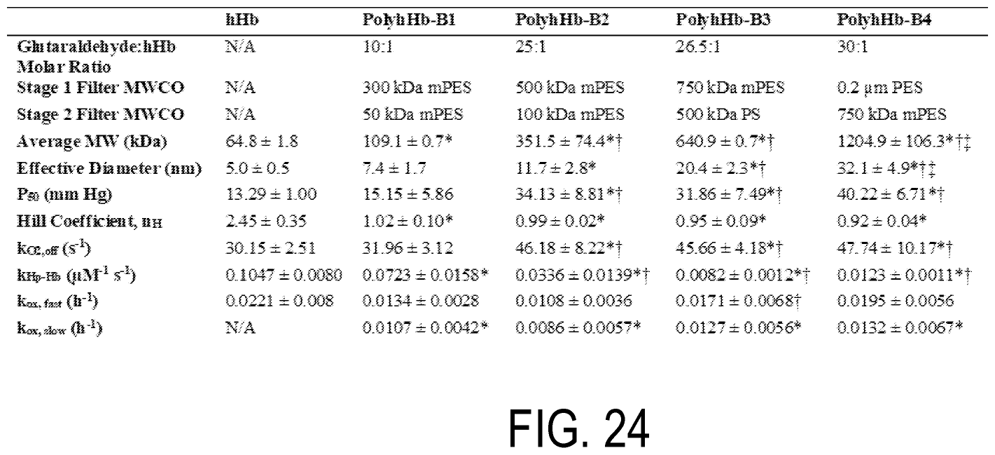

The P₅₀ (partial pressure of O₂ at which 50% of the hHb/PolyhHb is saturated with O₂) was measured using a Blood Oxygen Binding System. The P₅₀ for the fractions PolyB1, B2, B3, and B4 were 40.53 mm Hg, 34.78 mm Hg, 45.06 mm Hg, and 45.11 mm Hg, respectively. The P₅₀ for the PolyhHb was greater than that of unmodified hHb (12 mm Hg). This increase in P₅₀ is consistent with locking the heme in the tense (T) quaternary state conformation prior to synthesis. The resulting oxygen equilibrium curves for each fraction bracket (B1-B4) and free hHb are shown in FIG. 22A. In addition to the changes in P₅₀, polymerizing hHb led to a decrease in cooperativity (n, Hill coefficient; a value greater than 1 corresponds to facilitated O₂ binding) compared to unmodified hHb (n=2.5). PolyB1, B2, B3, and B4 possessed a Hill coefficient of 1.47, 1.01, 1.18, and 1.18, respectively. The B1 fraction had a higher cooperativity than the other PolyhHb fractions due to a larger percentage of hHb compared to the other fractions. The oxygen offloading kinetics for each PolyhHb fraction and free hHb are shown in FIG. 22B. The O₂ offloading rate constant (k_{off,O2}) for each of the fractions (B1, B2, B3, and B4) were 31.96±2.54 s-1, 40.48±3.28 s-1, 46.29±3.97 s-1, and 43.77±9.90 s⁻¹ compared to hHb (32.83 s-1). FIGS. 22C-22D shows the kinetics of hHb/PolyhHb binding to haptoglobin (Hp), where PolyB1, B2, B3, and B4 were found to quench a lower number of Hb binding sites in Hp compared to hHb, suggesting substantial removal of cell-free Hb and small hHb polymers from the PolyhHb solution. PolyB1, B2, B3, and B4 possessed a 2nd order (hHb/PolyhHb)-Hp binding rate constant (kHp-Hb) of 0.0725±0.0158 μM-1s⁻¹, 0.0433±0.0017 μM-1s⁻¹, 0.0082±0.0012 μM-1s⁻¹, and 0.0123±0.0011 μM-1s⁻¹, respectively which were drastically reduced compared to unmodified hHb (kHp-Hb=0.116 μM-1s⁻¹). Interestingly, PolyB3 and B4 had a lower Hp binding rate constant in comparison to that of PolyB1 and B2, likely due to both a higher degree of intermolecular and intramolecular crosslinking, which is consistent with the results from SEC-HPLC, DLS and SDS-PAGE (FIGS. 23A-23C).

The elution time of bracketed PolyhHb fractions was measured using size exclusion chromatography (SEC). This data is shown in FIG. 23A. Specifically, PolyB1, B2, B3, and B3 were found to elute at 8.92 mins, 8.65 mins, 8.40 mins, and 8.15 mins which corresponded to an average MW of 284.19 kDa, 448.50 kDa, 688.15 kDa, and 1049.93 kDa, respectively. FIG. 23B shows the particle size measured via dynamic light scattering (DLS) for each of the fractions compared with free hHb. PolyB1, B2, B3, and B4 were found to have an effective diameter of 7.4±1.4 nm, 12.5±2.2 nm, 21.4±2.2 nm, and 34.2±3.9 nm which is significantly larger than that of hHb (5.6 nm). On average, the polydispersity index (PDI) for the various fractions was less than 0.3. These results compare well with the effective MW measured with SEC-HPLC. The final check for polydispersity and average size based on MW was assessed with SDS-PAGE (FIG. 23C). As the fractions increased in size/MW from B1 to B4, there was a noticeable smear through the high MW species, indicating larger species due to the increasing crosslinking density. In addition, the bands at 17 kDa and 32 kDa, which correspond to the hHb monomer (a/p) and dimer (ap), become less pronounced moving from hHb to PolyB4. This trend shows the reduction of cell-free hHb and other low MW PolyhHb species with larger fractions (B2-B4) compared to the smallest fraction B1.

Other advantages which are obvious, and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

1. A perfusion solution comprising polymerized hemoglobin, wherein the perfusion solution comprises less than 5% by weight low molecular weight hemoglobin species, based on the total weight of the perfusion solution.

2. The perfusion solution of claim 1, wherein the low molecular weight hemoglobin species can have a molecular weight less than 300 kDa.

3. The perfusion solution of claim 1, wherein the polymerized hemoglobin is prepared by a process that comprises:
polymerizing hemoglobin; and
filtering the perfusion solution by ultrafiltration against a filtration membrane having a pore size that separates the low molecular weight hemoglobin species from the polymerized hemoglobin.

4. The perfusion solution of claim 3, wherein the filtration membrane is rated for retaining solutes having a molecular weight greater than a molecular weight of the low molecular weight species but less than a molecular weight of the polymerized hemoglobin, thereby forming a retentate fraction comprising the polymerized hemoglobin and a permeate fraction comprising the low molecular weight hemoglobin species.

5. The perfusion solution of claim 3, wherein the ultrafiltration comprises tangential-flow filtration.

6. The perfusion solution of claim 4, wherein the retentate fraction comprises the polymerized hemoglobin having a molecular weight of greater than 300 kDa and the permeate fraction comprises the low molecular weight hemoglobin species having a molecular weight of less than 300 kDa.

7. The perfusion solution of claim 6, wherein the polymerized hemoglobin is prepared by a process that further comprises filtering the retentate fraction comprising the polymerized hemoglobin by ultrafiltration against a second filtration membrane, thereby forming a second retentate fraction comprising the polymerized hemoglobin with a molecular weight above a cutoff value and a second permeate fraction comprising species having a molecular weight below the cutoff value and above 300 kDa.

8-12. (canceled)

13. The perfusion solution of claim 7, wherein the polymerized hemoglobin is prepared by a process that further comprises filtering the second retentate fraction comprising the polymerized hemoglobin by ultrafiltration against a third filtration membrane, thereby forming a third retentate fraction comprising high molecular weight impurities with a molecular weight above a second cutoff value and a third permeate fraction comprising the polymerized hemoglobin with a molecular weight below the second cutoff value and above the cutoff value.

14-18. (canceled)

19. The perfusion solution of claim 3, wherein the filtration membrane is rated for retaining solutes having a molecular weight greater than 0.2 μm, thereby forming a retentate fraction comprising species having a molecular weight of greater than 0.2 μm and a permeate fraction comprising the polymerized hemoglobin having a molecular weight of less than 0.2 μm and the low molecular weight hemoglobin species.

20. (canceled)

21. The perfusion solution of claim 1, wherein the polymerized hemoglobin is prepared by a process that further comprises filtering the permeate fraction comprising the polymerized hemoglobin and the low molecular weight hemoglobin species by ultrafiltration against a second filtration membrane, thereby forming a second retentate fraction comprising the polymerized hemoglobin having a molecular weight below 0.2 μm and above a cutoff value and a second permeate fraction comprising the low molecular weight hemoglobin species having a molecular weight below the cutoff value.

22-26. (canceled)

27. The perfusion solution of claim 1, wherein the low molecular weight hemoglobin species comprises unreacted hemoglobin, cell-free hemoglobin, and small hemoglobin polymers or any combination thereof.

28. The perfusion solution of claim 1, wherein the perfusion solution comprises 0.5-15 g/dL of the polymerized hemoglobin, 25-85 mM NaCl, 1-3 mM KCl, 6-20 mM KH₂PO₄, 20-70 mM sodium gluconate, 5-21 mM sodium lactate, 1-4 mM magnesium gluconate, 0.6-1.2 mM CaCl₂) dihydrate, 11-16 mM NaOH, 1-4 mM adenine, 2-8 mM dextrose, 0.5-3 mM glutathione, 2-8 mM HEPES, 1-4 mM ribose, 7-30 mM mannitol, 10-40 g/L hydroxyethyl starch, and 40-160 mg/dL N-acetyl-L-cysteine.

29. (canceled)

30. The perfusion solution of claim 1, wherein the perfusion solution comprises from 1% to 5% by weight albumin, based on total weight of the perfusion solution.

31. The perfusion solution of claim 1, wherein the perfusion solution has an osmolarity from 270 to 370 mOsm, such as an osmolarity from 324 to 346 mOsm.

32. The perfusion solution of claim 1, wherein the perfusion solution has a viscosity from 2 cP to 4.5 cP at normothermic conditions, such as a viscosity from 2.9 to 3.7 cP at normothermic conditions.

33. The perfusion solution of claim 1, wherein the perfusion solution has a colloid osmotic pressure from 14 mm Hg to 20 mm Hg, such as a colloid osmotic pressure from 16.8 to 17.6 mm Hg.

34. The perfusion solution of claim 1, wherein the polymerized hemoglobin is synthesized at a molar ratio from 20:1 to 40:1 of glutaraldehyde and hemoglobin.

35. (canceled)

36. The perfusion solution of claim 1, wherein the partial pressure of oxygen at which 50% of the polymerized hemoglobin is saturated with oxygen is from 1 mm Hg to 50 mm Hg, such as from 14 mm Hg to 16 mm Hg.

37. The perfusion solution of claim 1, wherein the polymerized hemoglobin exhibits an auto-oxidation rate constant at 37° C. from 0.0020 to 0.0085 h⁻¹, such as from 0.0045 to 0.0065 h⁻¹.

38. The perfusion solution of claim 1, wherein the perfusion solution further comprises a metabolic suppressant agent.
